(19) United States
(12) Patent Application Publication
(10) Pub. No.: US 2022/0008563 A1
(43) Pub. Date: Jan. 13, 2022

(54) RADIOLABELLED AND NONRADIOLABELLED PEGYLATED COMPOUNDS AND USES THEREOF

(71) Applicant: The Regents of the University of California, Oakland, CA (US)

(72) Inventors: Jogeshwar Mukherjee, Irvine, CA (US); Christopher Liang, Irvine, CA (US); Phuc Quang Lam, Irvine, CA (US); Rommani Mondal, Chino Hills, CA (US); Stephanie Martinez, Irvine, CA (US)

(21) Appl. No.: 17/372,410

(22) Filed: Jul. 9, 2021

Related U.S. Application Data
(60) Provisional application No. 63/050,731, filed on Jul. 10, 2020.

(51) Int. Cl. A61K 51/04; C07B 59/00; C07D 471/04; A61P 25/28; G01N 33/60

(52) U.S. Cl. CPC A61K 51/0455; C07B 59/002; G01N 33/60; A61P 25/28; C07D 471/04

(57) ABSTRACT

The disclosure provides compounds for detecting neurodegeneration and/or identifying and monitoring the progression of inflammation in neurodegenerative diseases. The disclosure further provides compounds that inhibit the activity of monoamine oxidases, and uses thereof.

### CROSS REFERENCE TO RELATED APPLICATIONS

This application claims priority under 35 U.S.C. § 119 from Provisional Application Ser. No. 63/050,731 filed Jul. 10, 2020, the disclosure of which is incorporated herein by reference.

### STATEMENT OF GOVERNMENT SUPPORT

This invention was made with Government support under Grant No. AG029479, awarded by the National Institutes of Health. The Government has certain rights in the invention.

### TECHNICAL FIELD

The disclosure provides compounds for detecting neurodegeneration and/or identifying and monitoring the progression of inflammation in neurodegenerative diseases. The disclosure further provides compounds that inhibit the activity of monoamine oxidases, and uses thereof.

### BACKGROUND

Neurodegenerative diseases affect millions of people worldwide. Alzheimer's disease and Parkinson's disease are the most common neurodegenerative diseases. In 2016, an estimated 5.4 million Americans were living with Alzheimer's disease. An estimated 930,000 people in the United States could be living with Parkinson's disease by 2020. Neurodegenerative diseases occur when nerve cells in the brain or peripheral nervous system lose function over time and ultimately die. Although treatments may help relieve some of the physical or mental symptoms associated with neurodegenerative diseases, there is currently no way to slow disease progression and no known cures. The risk of being affected by a neurodegenerative disease increases dramatically with age. More Americans living longer means more people may be affected by neurodegenerative diseases in coming decades. This situation creates a critical need to improve our understanding of what causes neurodegenerative diseases and develop new approaches for treatment and prevention. Scientists recognize that the combination of a person's genes and environment contributes to their risk of developing a neurodegenerative disease. That is, a person might have a gene that makes them more susceptible to a certain neurodegenerative disease. But whether, when, and how severely the person is affected depends on environmental exposures throughout life.

### SUMMARY

A series of compounds were developed and described herein. The compounds were used in studies of inflammation and neurodegeneration in Parkinson's disease models and complement studies of Aβ plaques and NFT in Alzheimer's disease. The compounds of the disclosure evaluated as a suitable agent for fluorine-18 radiolabeling. In order to assess the value of the compounds in AD imaging, the following studies and assays are carried out herein: (1) molecular modeling of the compounds binding to human AD Tau; (2) synthesis of compounds; (3) in vitro binding affinity of the compounds; (4) radiosynthesis of the compounds; (5) comparison of compounds to [¹²⁵I]IPPI in postmortem AD human brain slices; (6) evaluation of drug effects on the binding of the compounds in postmortem AD human brain slices; and (7) PET/CT studies of the compounds in normal mice.

Positron emission tomographic (PET) studies of amyloid β(Aβ) accumulation in Alzheimer's disease (AD) have shown clinical utility. Provided herein is the development and evaluation of the effectiveness of fluorine-18 radiotracer compounds for Aβ plaque imaging. Nucleophilic [¹⁸F]fluoride was used in a one-step radiosynthesis for the compounds. Using post mortem human AD brain tissues consisting of anterior cingulate (AC) and corpus callosum (CC), the binding affinity for one of the compounds ([¹⁸F]Flotaza) was found to have a Ki=1.68 nM for human Aβ plaques and weak (>10⁻⁵ M) for Tau protein. Radiosynthesis of the compounds of the disclosure was very efficient in radiochemical yields. For example, [¹⁸F]Flotaza was found to be efficiently made in high radiochemical yields (>25%) with specific activities >74 GBq/μmol. Brain slices from all AD subjects were positively immunostained with anti-Aβ. The ratio of the compounds disclosed herein in gray matter AC to white matter CC was found almost exclusively in the gray matter of the subjects. Very little white matter binding was seen. As such, compounds of the disclosure therefore find use as PET radiotracers for PET imaging studies of neurodegeneration (e.g., human Aβ plaques, Neurofibrillary tangles (NFTs), etc.).

In a particular embodiment, the disclosure provides a compound having the structure of Formula (I) or Formula (II):

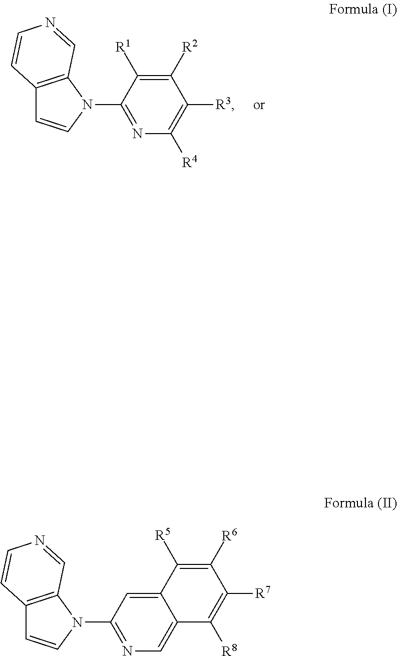

or a pharmaceutically acceptable salt, solvate, polymorph, or prodrug thereof, wherein, R¹-R⁴ are each individually selected from H, —OR⁹, a halogen, a radiohalogen, a hydroxyl, an amino, an alkoxy, an azide, a ketone, a carboxyl, a carboxylate, an aldehyde, a boronic acid, a boronic ester, a haloformyl, an imide, a nitrile, an isonitrile, a nitro, a thiol, a sulfinyl, a sulfo, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆) hetero-alkenyl, an optionally substituted (C₁-C₆)hetero-alkynyl, an optionally substituted (C₄-C₁₂)cycloalkyl, an aryl, and a heterocycle group, wherein at least one of R¹-R⁴ is —OR⁹; R⁵-R⁸ are each individually selected from H, —OR⁹, a halogen, a radiohalogen, a hydroxyl, an alkoxy, an azide, a ketone, a carboxyl, a carboxylate, an aldehyde, a boronic acid, a boronic ester, a haloformyl, an imide, a nitrile, an isonitrile, a nitro, a thiol, a sulfinyl, a sulfo, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆)hetero-alkenyl, an optionally substituted (C₁-C₆)hetero-alkynyl, an optionally substituted (C₄-C₁₂)cycloalkyl, an aryl, and a heterocycle group, wherein at least one of R⁵-R⁸ is —OR⁹ or a radiohalogen; R⁹ is an ether or polyether having the structure of

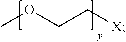

X is a halogen, a radiohalogen, or a hydroxyl; and y is an integer selected from 1, 2, 3, 4, 5, 6, 7, 8, 9 and 10. In another embodiment, R¹-R⁴ is selected from —OR⁹ and H, and one of R¹-R⁴ is —OR⁹. In yet another embodiment, R¹, R³ and R⁴ are H, and R² is —OR⁹. In a further embodiment, R¹-R⁴ is selected from —OR⁹, a radiohalogen, and H, and wherein one of R¹-R⁴ is —OR⁹ and one of R¹-R⁴ is ¹²⁵I. In yet a further embodiment, R¹ and R⁴ are H, R² is —OR⁹, and R³ is a radiohalogen. In a certain embodiment, R³ is ¹²⁵I. In another embodiment, R⁵-R⁸ is selected from ²¹¹At and H, and one of R⁵-R⁸ is a radiohalogen. In yet another embodiment, R⁵, R⁷, and R⁸ are H, and R⁶ is a radiohalogen. In a further embodiment, R⁶ is ¹²⁵I or ²¹¹At. In a further embodiment, R⁵-R⁸ is selected from —OR⁹ and H, and one of R⁵-R⁸ is —OR⁹. In yet a further embodiment, X is a radiohalogen selected from ¹⁸F, ¹²⁵I, ²¹¹At, ¹²³I, ¹²⁴I, and ^{76/77/78}Br. In another embodiment, X is ¹⁸F. In yet another embodiment, the compound comprises the structure of:

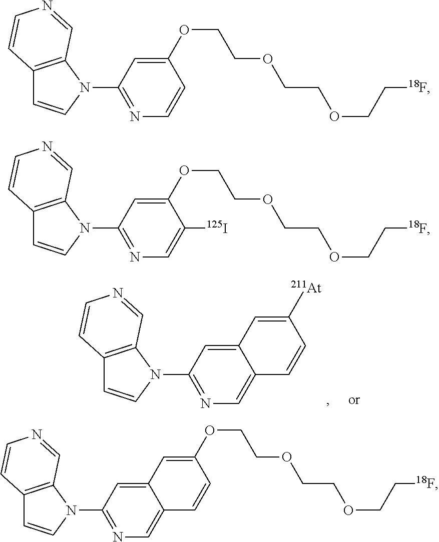

or a pharmaceutically acceptable salt, solvate, polymorph, or prodrug thereof.

In a particular embodiment, the disclosure also provides a method of imaging neurodegeneration in a postmortem brain specimen, comprising: contacting a postmortem brain specimen from a subject who has a neurodegenerative disorder or is suspected of having a neurodegenerative disorder with a compound of the disclosure, detecting and/or quantitating binding of the compound to brain tissue comprising neurodegeneration using an imaging technique that detects radioactivity emitted by the compound.

In a certain embodiment, the disclosure further provides a pharmaceutical composition comprising a compound disclosed herein and a pharmaceutically acceptable carrier, diluent, and/or excipient, wherein the compound comprises a radiohalogen. In a further embodiment, the pharmaceutical composition is formulated for oral or parenteral delivery.

In a particular embodiment, the disclosure further provides a method for imaging neurodegeneration in a subject having a neurodegenerative disorder or suspected of having a neurodegenerative disorder, comprising: administering the pharmaceutical composition of the disclosure to the subject, detecting and/or quantitating binding of the compound to brain tissue comprising neurodegeneration using an imaging technique that detects radioactivity emitted by the compound. In a further embodiment, the subject has neurodegenerative disorder selected from the group consisting of Alzheimer's disease, Parkinson's disease, prion disease, motor neuron diseases, Huntington's disease, spinocerebellar ataxia, spinal muscular atrophy, amyotrophic lateral sclerosis, Friedreich's ataxia, and Lewy body disease. In another embodiment, the imaging technique is selected from positron emission tomography (PET) imaging, single photon emission computed tomography (SPECT), magnetic resonance imaging, or autoradiography.

In a certain embodiment, the disclosure also provides a compound having the structure of Formula (III):

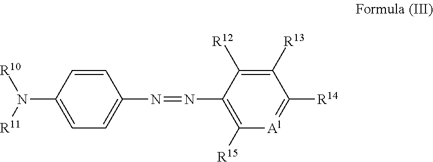

or a pharmaceutically acceptable salt, solvate, polymorph, or prodrug thereof, wherein, A¹ is selected from N, or CR¹⁶; R¹⁰ and R¹¹ are individually selected from H or a (C₁-C₆)alkyl; R¹²-R¹⁶ are each individually selected from H, —OR¹⁷, a halogen, a radiohalogen, a hydroxyl, an amino, an alkoxy, an azide, a ketone, a carboxyl, a carboxylate, an aldehyde, a boronic acid, a boronic ester, a haloformyl, an imide, a nitrile, an isonitrile, a nitro, a thiol, a sulfinyl, a sulfo, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆) hetero-alkenyl, an optionally substituted (C₁-C₆)hetero-alkynyl, an optionally substituted (C₄-C₁₂)cycloalkyl, an aryl, and a heterocycle group, wherein at least one of R¹-R⁴ is —OR¹⁷; R¹⁷ is an ether or polyether having the structure of

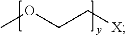

X is a halogen, a radiohalogen or a hydroxyl; and y is an integer selected from 1, 2, 3, 4, 5, 6, 7, 8, 9 and 10.

The details of one or more embodiments of the disclosure are set forth in the accompanying drawings and the description below. Other features, objects, and advantages will be apparent from the description and drawings, and from the claims.

### DESCRIPTION OF DRAWINGS

FIG. 1 provides examples of known Tau binding radioligands: [¹⁸F]THK5351; [¹⁸F]Flortaucipir (T807); [¹⁸F]RO6958948; and [¹⁸F]MK-6240.

FIG. 2 presents embodiments of fluoroalkylazaindoles (FAZIN)-based compounds, namely fluorine-18 pegylated meta azaindole based compounds.

FIG. 3 presents additional embodiments of FAZIN-based compounds of the disclosure, namely fluorine-18 pegylated para azaindoles of the disclosure.

FIG. 4 presents embodiments of FAZIN-based compounds of the disclosure, namely fluorine-18 pegylated ortho azaindoles of the disclosure.

FIG. 5 presents embodiments of FAZIN-based compounds of the disclosure, namely fluorine-18 pegylated N-ortho azaindoles of the disclosure.

FIG. 6 presents a synthesis scheme for producing FAZIN3. 2-Chloro-4-hydroxypyridine was reacted with 2-[2-(2-bromoethoxy)ethoxy]ethanol (bromo-PEG₃-alcohol) to provide the PEG₃-alcohol derivative of 2-chloro-4-PEG₃-OH-pyridine derivative. This was reacted with azaindole to provide the azaindole-PEG₃-OH derivative. The alcohol was fluorinated using diethylamino sulfur trifluoride (DAST) to provide FAZIN3.

FIG. 7 provides for the radiosynthesis of [¹⁸E]FAZIN3: Azaindole-PEG₃-OH was converted to the corresponding tosylate. The tosylate was reacted with fluorine-18 to provide [¹⁸F]FAZIN3, which was characterized chromatographically and used in the in vitro and in vivo studies.

FIG. 8 presents the synthesis scheme for IPPI and BrPPI. Azaindole 1 was reacted with 3-chloro-6-iodoisoquinoline 2 in dimethylformamide (DMF) in the presence of potassium tert-butoxide for 24 hours at 120° C. to provide IPPI, 5. Similarly, azaindole 1 was reacted with 3-fluoro-6-bromoisoquinoline 3 in DMF in the presence of potassium tert-butoxide for 24 hours at 120° C. to provide 6-bromo-3-(1H-pyrrolo[2,3-c]pyridine-1-yl)isoquinoline, BrPPI, 4.

FIG. 9 presents the radiosynthesis of [¹²⁵I]IPPI. BrPPI, 4 was held at reflux with bis(tributyltin) in the presence of tetrakis(triphenylphosphine)palladium(0) for 24 hours to provide 6-tributyltin-3-(1H-pyrrolo[2,3-c]pyridine-1-yl)isoquinoline, 6. Tin precursor 6 was reacted with sodium [¹²⁵I]iodide under oxidative conditions using peracetic acid to provide [¹²⁵I]IPPI. Thin layer chromatogram of [¹²⁵I]IPPI shows purity of >95%.

FIG. 10 demonstrates the one step radiosynthesis of ¹²⁵I-IAZA and ¹²⁴ I-IAZA. Tributyltin-4′-N,N-dimethylaminoazobenzene precursor 6 is reacted with the sodium-[¹²⁴I]iodide under oxidative conditions to form ¹²⁴I-IPPI.

FIG. 11 presents additional embodiments of compounds of the disclosure, namely pegylated derivatives of isoquinoline derivatives.

FIG. 12 presents additional embodiments of compounds of the disclosure, namely additional Astatine-211 derivatives of IPPI.

FIG. 13 presents additional embodiments of compounds of the disclosure, namely pegylated derivatives with radiohalogens.

FIG. 14 presents a chromatogram from thin layer chromatography showing the high degree of product formation for [¹⁸F]FAZIN3.

FIG. 15 provides an in vivo scan of the body distribution of [¹⁸F]FAZIN3 in a normal C57B/6 mouse. Orthogonal view of mouse PET/CT 30 minute summed images after intraperitoneal injection of 50 μCi [¹⁸F]FAZIN3. Brain uptake is observed, with little defluorination as evidenced from a lack of bone uptake. [18F]FAZIN3 seems to clear from the liver to the lower GI tract as well as to the urinary bladder.

FIG. 16A-D presents the results of the use of [¹⁸F]FAZIN3 in Alzheimer's disease tissue. (A-D) The gray matter and white matter were clearly delineated in the autoradiographic images. All control subjects exhibited lower [¹⁸F]FAZIN3 binding in anterior cingulate tissue compared to the Alzheimer's disease subjects. In the case of the Alzheimer's disease subjects, anterior cingulate tissue exhibited a more than 2-fold increase in binding of [¹⁸F]FAZIN3 (AC/CC=2.27).

FIG. 17A-D presents the results of the use of [¹⁸F]FAZIN3 in Parkinson's disease tissue. (A-D) The gray matter and white matter were clearly delineated in the autoradiographic images. All control subjects exhibited lower [¹⁸F]FAZIN3 binding in the anterior cingulate tissue compared to Parkinson Disease subjects. In the case of the Parkinson Disease subjects, anterior cingulate tissue exhibited a more than 2-fold increase in binding of [¹⁸F]FAZIN3 (PD/CN=2.12).

FIG. 18A-H: provides the results of [¹²⁵I]IPPI competitive binding assay with various inhibitors. (A) Postmortem human brain 10 μm thick sections (from two AD subjects, 11-107 and 11-38) of anterior cingulate (AC) grey matter (arrows showing GM) and corpus callosum (CC) white matter (WM). (B) [¹²⁵I]IPPI binding to grey matter, AC with low nonspecific binding in white matter, CC. (C) Deprenyl, 1 μM effect on [¹²⁵I]IPPI binding. (D) Clorgyline, 10 μM effect on [¹²⁵I]IPPI binding. (E) BrPPI, 10 μM effect on [¹²⁵I ]IPPI binding. (F) MK-6240, 10μM effect on [¹²⁵I]IPPI binding. (G) Immunostaining of 11-38 with Dako A0024 for total Tau; (i) picture of entire slice and (ii) magnification (x10) of box in (i). (H) Plot comparing GM and WM in the two AD subjects with different drugs.

FIG. 19 provides the results of [¹⁸F]FAZIN3 competitive binding assay with MAO-A and MAO-B inhibitors. Clorgyline displaced more than 80% of [¹⁸F]FAZIN3 suggesting binding to MAO-A. Deprenyl had little effect on [¹⁸F]FAZIN3, suggesting little effect on MAO-B.

FIG. 20 shows that [¹²⁴I]IPPI bound to Alzheimer's disease brain grey matter containing neurofibrillary tangles in postmortem brain slices.

FIG. 21 demonstrates the one step radiosynthesis of ¹²⁵I-IAZA and ¹²⁴ I-IAZA from 4-Tributyltin-4′-N,N-dimethylaminoazobenzene precursor 10. 4-Tributyltin-4′-N,N-dimethylaminoazobenzene precursor 10 was synthesized using 4-iodo-4′-N,N-dimethylaminoazobenze. The radiosynthesis of [¹²⁵I]IAZA and [¹²⁴I]IAZA was carried out using electrophilic substitution by sodium-[¹²⁵]iodide or sodium-[₁₂₄]iodide. Thin layer radio-chromatography of purified ¹²⁵I-IAZA confirmed radiochemical purity.

FIG. 22A-F provides the results of brain autoradiography in postmortem human brain from an Alzheimer patient using ¹²⁵I-IAZA and ¹²⁴I-IAZA. (A) AD brain slice showing gray matter (GM), anterior cingulate and white matter (WM) corpus callosum. (B)-(C)¹²⁵I-IAZA binding in the gray matter regions in adjacent slices, consistent with the presence of AB plaques. (D) AD brain cortex slice showing gray matter on the left edges. (E)-(F) ¹²⁵I-IAZA binding in the outer regions containing gray matter which is consistent with Tau buildup.

FIG. 23 demonstrates the radiosynthesis of [¹⁸F]Flotaza. 4-hydroxy-4′-dimethylaminoazobenzene 7 was reacted with bromo-PEG₃-alcohol (Br(CH₂CH₂O)₃H) in dimethylformamide (DMF) in the presence of potassium tert-butoxide (K⁺O^{t}Bu). Tosylate 9 was obtained by reacting toluenesulfonyl chloride (TsCl) with 2-{2-[2-hydroxyethoxy]ethoxy}ethoxy)-4′-N,N-dimethylaminoazobenzene 8 in dichloromethane (CH₂Cl₂). Flotaza was prepared by reacting tosylate 9 with [¹⁸F]fluoride, Kryptofix and potassium carbonate (K₂CO₃) in acetonitrile (CH₃CN) to provide [¹⁸F]Flotaza. RadioTLC confirmed radiochemical purity of >95% for [¹⁸F]Flotaza (bottom panel) and was obtained in amounts of 370 to 740 MBq in specific activities generally >37 TBq/mmol.

FIG. 24A-E provides the results of brain autoradiography in postmortem human brain from an Alzheimer patient using [¹⁸F]Flotaza. (A) AD brain slice showing gray matter (GM), anterior cingulate and white matter (WM) corpus callosum. (B) Anti-Ab immunostained adjacent section showing presence of Ab plaques (inset at x40 magnification). (C)[¹⁸F]Flotaza binding in the gray matter regions in adjacent slices, consistent with the presence of Ab plaques. (D) High levels of [¹⁸F]Flotaza binding in gray matter in six AD subjects with very little white matter binding. (E) A 5 mm long plot through cortex (red lines shown in C) showing high amounts of [¹⁸F]Flotaza in the outer layers of the cortex, with almost background levels in white matter.

FIG. 25A-F demonstrates the results of using [¹⁸F]Flotaza for Aβ plaques and [¹²⁵I]IPPI NFT in same AD subject. (A) Anti-Aβ immunostained section showing presence of Aβ plaques (×4 magnification, C). (B) [¹⁸F]Flotaza binding in the anterior cingulate in adjacent slices, consistent with the presence of Aβ plaques. (D) Anti-Tau immunostained section showing presence of total Tau protein (×4 magnification, F). (E) ^{[125}I]IPPI binding in the anterior cingulate in adjacent slices, consistent with the presence of NFT.

### DETAILED DESCRIPTION

As used herein and in the appended claims, the singular forms “a,” “an,” and “the” include plural referents unless the context clearly dictates otherwise. Thus, for example, reference to “a MAO inhibitor” includes a plurality of such MAO inhibitors and reference to “the imaging agent” includes reference to one or more imaging agents and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this invention is not limited to the particular methodology, protocols, and reagents, etc., described herein and as such may vary. The terminology used herein is for the purpose of describing particular embodiments only and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%.

The term “alkenyl”, refers to an organic group that is comprised of carbon and hydrogen atoms that contains at least one double covalent bond between two carbons. Typically, an “alkenyl” as used in this disclosure, refers to organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkenyl can form a double bond to a carbon of a parent chain, an alkenyl group of three or more carbons can contain more than one double bond. In certain instances, the alkenyl group will be conjugated, in other cases an alkenyl group will not be conjugated, and yet other cases the alkenyl group may have stretches of conjugation and stretches of nonconjugation. Additionally, if there is more than 2 carbon, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted, unless stated otherwise.

The term “alkyl”, refers to an organic group that is comprised of carbon and hydrogen atoms that contains single covalent bonds between carbons. Typically, an “alkyl” as used in this disclosure, refers to an organic group that contains 1, 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. Where if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted, unless stated otherwise.

The term “alkynyl”, refers to an organic group that is comprised of carbon and hydrogen atoms that contains a triple covalent bond between two carbons. Typically, an “alkynyl” as used in this disclosure, refers to organic group that contains that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkynyl can form a triple bond to a carbon of a parent chain, an alkynyl group of three or more carbons can contain more than one triple bond. Where if there is more than 3 carbon, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted, unless stated otherwise.

The term “aryl”, as used in this disclosure, refers to a conjugated planar ring system with delocalized pi electron clouds that contain only carbon as ring atoms. An “aryl” for the purposes of this disclosure encompass from 1 to 5 aryl rings wherein when the aryl is greater than 1 ring the aryl rings are joined so that they are linked, fused, or a combination thereof. An aryl may be substituted or unsubstituted, or in the case of more than one aryl ring, one or more rings may be unsubstituted, one or more rings may be substituted, or a combination thereof.

The term generally represented by the notation “Cₓ-C_{y}” (where x and y are whole integers and y>x) prior to a functional group, e.g., “C₁-C₁₂ alkyl” refers to a number range of carbon atoms. For the purposes of this disclosure any range specified by “Cₓ-C_{y}” (where x and y are whole integers and y>x) is not exclusive to the expressed range, but is inclusive of all possible ranges that include and fall within the range specified by “Cₓ-C_{y}” (where x and y are whole integers and y>x). For example, the term “C₁-C₄” provides express support for a range of 1 to 4 carbon atoms, but further provides implicit support for ranges encompassed by 1 to 4 carbon atoms, such as 1 to 2 carbon atoms, 1 to 3 carbon atoms, 2 to 3 carbon atoms, 2 to 4 carbon atoms, and 3 to 4 carbon atoms.

The term “cycloalkenyl”, as used in this disclosure, refers to an alkene that contains at least 4 carbon atoms but no more than 12 carbon atoms connected so that it forms a ring. A “cycloalkenyl” for the purposes of this disclosure encompasses from 1 to 4 cycloalkenyl rings, wherein when the cycloalkenyl is greater than 1 ring, then the cycloalkenyl rings are joined so that they are linked, fused, or a combination thereof. A cycloalkenyl may be substituted or unsubstituted, or in the case of more than one cycloalkenyl ring, one or more rings may be unsubstituted, one or more rings may be substituted, or a combination thereof.

The term “cylcoalkyl”, as used in this disclosure, refers to an alkyl that contains at least 3 carbon atoms but no more than 12 carbon atoms connected so that it forms a ring. A “cycloalkyl” for the purposes of this disclosure encompasses from 1 to 4 cycloalkyl rings, wherein when the cycloalkyl is greater than 1 ring, then the cycloalkyl rings are joined so that they are linked, fused, or a combination thereof. A cycloalkyl may be substituted or unsubstituted, or in the case of more than one cycloalkyl ring, one or more rings may be unsubstituted, one or more rings may be substituted, or a combination thereof.

The term “hetero-” when used as a prefix, such as, hetero-alkyl, hetero-alkenyl, hetero-alkynyl, or hetero-hydrocarbon, for the purpose of this disclosure refers to the specified hydrocarbon having one or more carbon atoms replaced by non-carbon atoms as part of the parent chain. Examples of such non-carbon atoms include, but are not limited to, N, O, S, Si, Al, B, and P. If there is more than one non-carbon atom in the hetero-based parent chain then this atom may be the same element or may be a combination of different elements, such as N and O.

The term “heterocycle”, as used in this disclosure, refers to univalent radical ring structures that contain at least 1 non-carbon ring atom, and typically comprise from 3 to 12 ring atoms. A “heterocycle” for the purposes of this disclosure encompasses from 1 to 12 heterocycle rings wherein when the heterocycle is greater than 1 ring then the rings are joined so that they are linked, fused, or a combination thereof. A heterocycle may be a hetero-aryl or nonaromatic, or in the case of more than one heterocycle ring, one or more rings may be nonaromatic, one or more rings may be hetero-aryls, or a combination thereof. A heterocycle may be substituted or unsubstituted, or in the case of more than one heterocycle ring one or more rings may be unsubstituted, one or more rings may be substituted, or a combination thereof. Typically, the non-carbon ring atom is N, O, S, Si, Al, B, or P. In case where there is more than one non-carbon ring atom, these non-carbon ring atoms can either be the same element, or combination of different elements, such as N and O. Examples of heterocycles include, but are not limited to: a monocyclic heterocycle such as, aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide; and polycyclic heterocycles such as, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine. In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

The terms “heterocyclic group”, “heterocyclic moiety”, “heterocyclic”, or “heterocyclo” used alone or as a suffix or prefix, refers to a heterocycle that has had one or more hydrogens removed therefrom.

The term “hetero-aryl” used alone or as a suffix or prefix, refers to a heterocycle or heterocyclyl having aromatic character. Examples of heteroaryls include, but are not limited to, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

The term “hetero-” when used as a prefix, such as, hetero-alkyl, hetero-alkenyl, hetero-alkynyl, or hetero-hydrocarbon, for the purpose of this disclosure refers to the specified hydrocarbon having one or more carbon atoms replaced by non-carbon atoms as part of the parent chain. Examples of such non-carbon atoms include, but are not limited to, N, O, S, Si, Al, B, and P. If there is more than one non-carbon atom in the hetero-based parent chain then this atom may be the same element or may be a combination of different elements, such as N and O.

The term “parenteral administration” and “administered parenterally” as used in this disclosure, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The term “pharmaceutically acceptable” as used in this disclosure, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salt” as used in this disclosure, refers to pharmaceutically acceptable, organic or inorganic acid or base salt of a compound of the disclosure. Representative pharmaceutically acceptable salts include, e.g., alkali metal salts, alkali earth salts, ammonium salts, water-soluble and water-insoluble salts, such as the acetate, amsonate (4,4-diaminostilbene-2, 2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The term “substituent” refers to an atom or group of atoms substituted in place of a hydrogen atom, e.g., a boronic acid group. For purposes of this invention, a substituent would include deuterium atoms. Examples of substituents include, but are not limited to, halo (e.g., F, Cl, Br or I), optionally substituted oxygen containing functional group (e.g., alcohol, ketone, aldehyde, acyl halide, carbonate, carboxylic acid, ester, and ether), optionally substituted nitrogen containing functional group (e.g., amide, amine, imine, azide, cyanate, azo, nitrate, nitrile, nitro, and nitroso), optionally substituted sulfur containing functional group (e.g., thiol, sulfide, disulfide, sulfoxide, sulfone, sulfinic acid, sulfonic acid, thiocyanate, thione, and thial), optionally substituted phosphorous containing functional group (e.g., phosphine, phosphonic acid, phosphate, phosphodiester), optionally substituted boron containing functional group (e.g., boronic acid, and boronic ester). Further examples of substituents include, but are not limited to, aryl, heterocycle, alkyl, heteroalkyl, alkenyl, heteroalkenyl, alkynyl, heteroalkynyl, cycloalkyl, alkoxy, ester, halo, hydroxyl, anhydride, carbonyl, carboxyl, carbonate, carboxylate, aldehyde, boronic acid, boronic ester, haloformyl, ester, hydroperoxy, peroxy, ether, orthoester, carboxamide, amine, imine, imide, azide, azo, cyanate, isocyanate, nitrate, nitrite, isonitrile, nitroso, nitro, nitrosooxy, pyridyl, sulfide, disulfide, sulfinyl, sulfo, thiocyanate, isothiocyanate, carbonothioyl, phosphino, phosphono, and phosphate.

The term “substituted” with respect to hydrocarbons, heterocycles, and the like, refers to structures wherein the parent chain contains one or more substituents.

The term “unsubstituted” with respect to hydrocarbons, heterocycles, and the like, refers to structures wherein the parent chain contains no substituents.

The terms “patient”, “subject” and “individual” are used interchangeably herein, and refer to an animal, particularly a human, to whom treatment including prophylaxis treatment is provided. This includes human and non-human animals. The term “non-human human animals” and “non-human mammals” are used interchangeably herein includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. A subject can be male or female. A subject can be a fully developed subject (e.g., an adult) or a subject undergoing the developmental process (e.g., a child, infant or fetus).

Inflammation in the brain, accumulation of amyloid β (Aβ) plaques and aggregation of neurofibrillary tangles (NFT) are three major pathological features Alzheimer's disease (AD). Positron emission tomography (PET) radiotracers are currently being used been for imaging of Aβ plaques and some of them are currently in clinical use. Significant efforts have been underway on the development and use of NFT PET imaging agents. Current data suggests that NFT imaging may be more precise in the evaluation of AD progression compared to Aβ plaque imaging. Selective PET quantification methods are currently under development for imaging NFT for staging of AD.

Imaging efforts have been underway to study microglial activation and astrocytosis, both of which contribute to neuroinflammation in the AD brain. Several PET radiotracers have been developed for the translocator protein (TSPO) to study microglia. However, reliable PET quantification of TSPO for use in staging neuroinflammation in AD subjects has been difficult due to various factors. These include low signal-to-noise ratios, low brain uptake, vasculature binding, activated microglia in mild cognitive impairment (MCI) versus astrocytes in AD and genotype stratification. Astrocytes have been targeted by imaging monoamine oxidase (MAO) which has been found to be elevated in AD and hence a target for therapeutic drug development. Comparative studies of the MAO-B radiotracer [¹¹C]DED and [¹⁸F]THK/[¹⁸F]T807 show a relationship between the two in AD.

Parkinson's disease (PD) is a movement disorder, characterized by: loss of dopaminergic terminals, and development of Lewy bodies. Growing evidence points to a critical role of a-synuclein aggregates as a potential trigger for Lewy bodies and PD. Although several neurotransmitter receptor systems, particularly dopamine, there is continued emphasis on the search for an early biomarker of neurodegeneration in PD. Targets for imaging the dopaminergic system are currently being pursued, and include presynaptic dopamine synthesis [¹⁸F]FDOPA, dopamine transporter (DAT) [¹⁸F]FPCIT, postsynaptic dopamine D2/D3 receptors [¹⁸F]fallypride and [¹⁸F]DMFP. Targets for imaging the cholinergic system, include a4b2 nicotinic receptor using [¹⁸F]Nifene and [¹⁸F]2FA85380. Tau imaging agents, like [¹⁸F]Flutaucipir([18F]T807) have been used to look for protein aggregates.

Two enzymes, MAO-A and MAO-B play a critical role in deamination of aminergic neurotransmitters (e.g., dopamine, norepinephrine) and peripheral tyramines. Inhibition of MAO to increase CNS dopamine levels has been pursued for drug development. There are irreversible and reversible inhibitors for MAO-A and MAO-B, and some of these drugs have been used for treatment of PD. Because MAO-A is considered a target for activated microglia and a marker for inflammation, ¹⁸F reversible inhibitors for human MAO-A were developed and described herein.

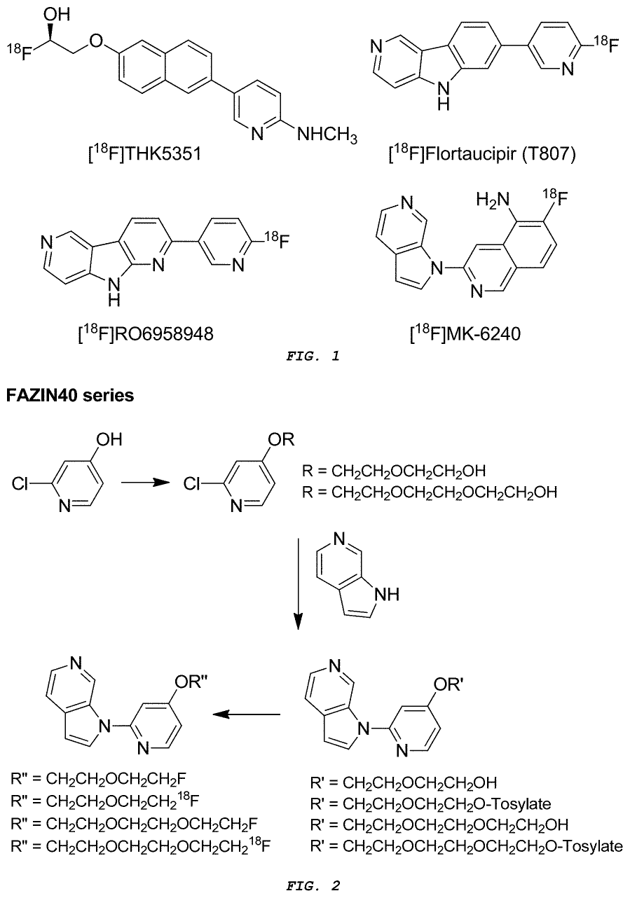

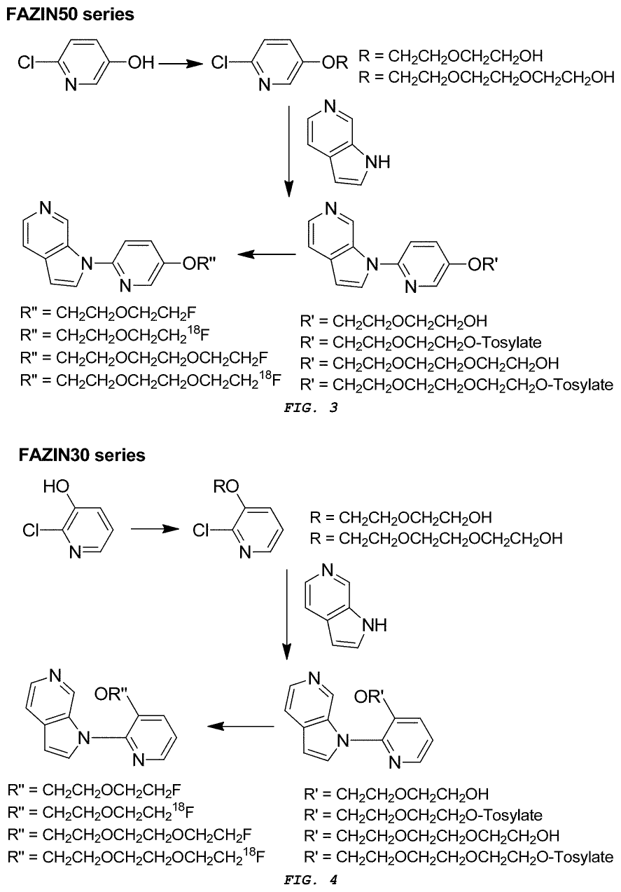

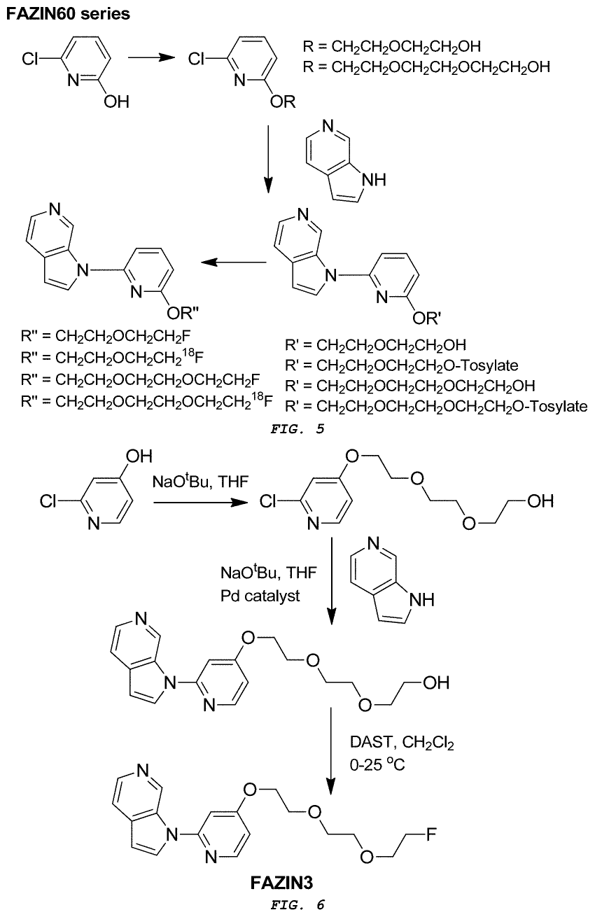

Both NFT and MAO-B have been imaged simultaneously with PET radiotracers such as [¹⁸F]THK5351 (see FIG. 1 at 2) in AD patients. Similarly, another NFT PET radiotracer [¹⁸F]T807 (see FIG. 1 at 3) shows MAO B binding. The related [¹⁸F]RO6958948 (see FIG. 1 at 4) continue for NFT imaging in AD, but MAO B binding has yet to be ascertained. A second generation of NFT PET radiotracers based on azaindole structure, such as [¹⁸F]MK-6240, have been developed which bind to Tau protein more selectively and off-target binding to MAO using in vivo may not be a concern. These have high affinity and are capable of crossing the brain-blood barrier to be valuable for the application of in vivo NFT imaging in PET studies. As a NFT radiotracer, [¹⁸F]MK-6240 shows characteristic changes in the brains of patients with AD. Autoradiographic studies, [¹⁸F]MK-6240 has some propensity of being displaced by MAO inhibitors in vitro. The studies presented herein using the azaindole [¹²⁵I ]IPPI have also shown resistance to MAO inhibitors at <1 μM concentrations.

In an effort to explore the possibility of developing reversible MAO inhibitors and to further understand the selectivity of the azaindole derivatives towards NFT and MAO, a series of compounds were developed and described herein. The compounds were used in studies of inflammation and neurodegeneration in Parkinson's disease models and complement studies of Aβ plaques and NFT in Alzheimer's disease. Computational modeling of the compounds with increasing length of ethylene glycol were carried out on AD Tau.

In a particular embodiment, the disclosure provides for a compound having the structure of Formula (I):

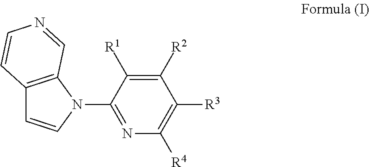

or a pharmaceutically acceptable salt, solvate, polymorph, or prodrug thereof, wherein,

R¹-R⁴ are each individually selected from H, —OR⁹, a halogen, a radiohalogen, a hydroxyl, an amino, an alkoxy, an azide, an anhydride, a carbonyl, a carboxyl, a carbonate, a carboxylate, an aldehyde, a boronic acid, a boronic ester, a haloformyl, a hydroperoxy, a peroxy, an ether, an orthoester, a carboxamide, an amine, an imine, an imide, an azo, a cyanate, an isocyanate, a nitrate, a nitrite, an isonitrile, a nitroso, a nitro, a nitrosooxy, a pyridyl, a sulfide, a disulfide, a sulfinyl, a sulfo, a thiocyanate, an isothiocyanate, a carbonothioyl, a phosphino, a phosphono, a phosphate, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆)hetero-alkenyl, an optionally substituted (C₁-C₆)hetero-alkynyl, an optionally substituted (C₄-C₁₂)cycloalkyl, an aryl, and a heterocycle group, wherein at least one of R¹-R⁴ is —OR⁹;

R⁹ is an alkoxy having the structure of

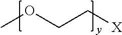

X is a halogen, a radiohalogen, or a hydroxyl; and

y is an integer selected from 1, 2, 3, 4, 5, 6, 7, 8, 9 and 10.

In a further embodiment, the disclosure provides for a compound having the structure of Formula I(a):

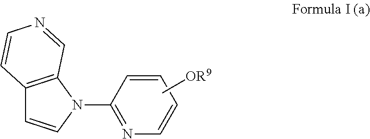

or a pharmaceutically acceptable salt, solvate, polymorph, or prodrug thereof, wherein,

R⁹ is an alkoxy having the structure of

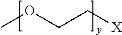

X is a halogen, a radiohalogen, or a hydroxyl; and

y is an integer selected from 1, 2, 3, 4, 5, 6, 7, 8, 9 and 10.

In yet a further embodiment, the disclosure provides for a compound having the structure of Formula I(b):

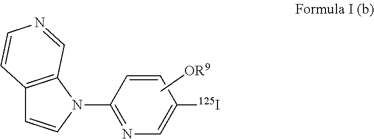

or a pharmaceutically acceptable salt, solvate, polymorph, or prodrug thereof, wherein,

R⁹ is an alkoxy having the structure of

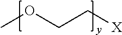

X is a halogen, a radiohalogen, or a hydroxyl; and

y is an integer selected from 1, 2, 3, 4, 5, 6, 7, 8, 9 and 10.

In a particular embodiment, the disclosure provides for a compound having the structure of:

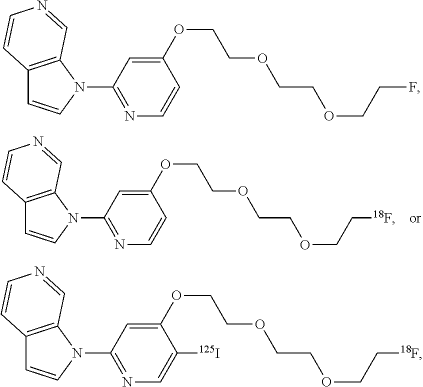

or a pharmaceutically acceptable salt, solvate, polymorph, or prodrug thereof.

In another embodiment, the disclosure provides for a compound having the structure of Formula II:

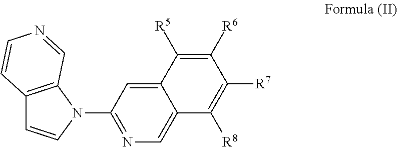

or a pharmaceutically acceptable salt, solvate, polymorph, or prodrug thereof, wherein,

R⁵-R⁸ are each individually selected from H, —OR⁹, a halogen, a radiohalogen, a hydroxyl, an amino, an alkoxy, an azide, an anhydride, a carbonyl, a carboxyl, a carbonate, a carboxylate, an aldehyde, a boronic acid, a boronic ester, a haloformyl, a hydroperoxy, a peroxy, an ether, an orthoester, a carboxamide, an amine, an imine, an imide, an azo, a cyanate, an isocyanate, a nitrate, a nitrite, an isonitrile, a nitroso, a nitro, a nitrosooxy, a pyridyl, a sulfide, a disulfide, a sulfinyl, a sulfo, a thiocyanate, an isothiocyanate, a carbonothioyl, a phosphino, a phosphono, a phosphate, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆) hetero-alkenyl, an optionally substituted (C₁-C₆)hetero-alkynyl, an optionally substituted (C₄-C₁₂)cycloalkyl, an aryl, and a heterocycle group, wherein at least one of R⁵-R⁸ is —OR⁹ or a radiohalogen;

R⁹ is an alkoxy having the structure of

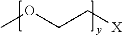

X is a halogen, a radiohalogen, or a hydroxyl; and

y is an integer selected from 1, 2, 3, 4, 5, 6, 7, 8, 9 and 10.

In a further embodiment, the disclosure provides for a compound having the structure of Formula II(a):

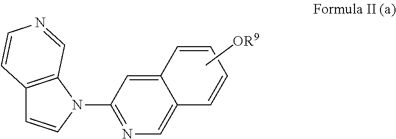

or a pharmaceutically acceptable salt, solvate, polymorph, or prodrug thereof, wherein,

R⁹ is an alkoxy having the structure of

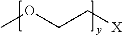

X is a halogen, a radiohalogen, or a hydroxyl; and

y is an integer selected from 1, 2, 3, 4, 5, 6, 7, 8, 9 and 10.

In yet a further embodiment, the disclosure provides for a compound having the structure of Formula II(b):

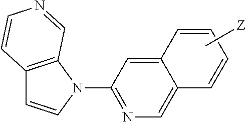

or a pharmaceutically acceptable salt, solvate, polymorph, or prodrug thereof, wherein,

Z is a radiohalogen.

In a particular embodiment, the disclosure provides for a compound having the structure of:

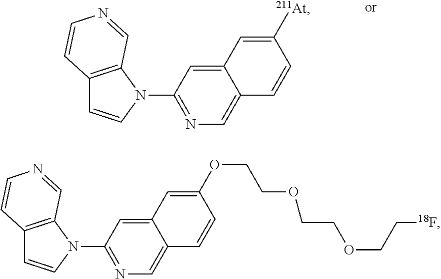

or a pharmaceutically acceptable salt, solvate, polymorph, or prodrug thereof.

In another embodiment, the disclosure provides for a compound having the structure of Formula (III):

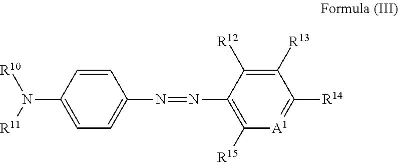

or a pharmaceutically acceptable salt, solvate, polymorph, or prodrug thereof, wherein,

A¹ is selected from N, or CR¹⁶;

R¹⁰ and R¹¹ are individually selected from H or a (C₁-C₆)alkyl;

R¹²-R¹⁶ are each individually selected from H, —OR¹⁷, a halogen, a radiohalogen, a hydroxyl, an amino, an alkoxy, an azide, a ketone, a carboxyl, a carboxylate, an aldehyde, a boronic acid, a boronic ester, a haloformyl, an imide, a nitrile, an isonitrile, a nitro, a thiol, a sulfinyl, a sulfo, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆) hetero-alkenyl, an optionally substituted (C₁-C₆)hetero-alkynyl, an optionally substituted (C₄-C₁₂)cycloalkyl, an aryl, and a heterocycle group, wherein at least one of R¹-R⁴ is —OR¹⁷;

R¹⁷ is an ether or polyether having the structure of

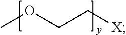

X is a halogen, a radiohalogen or a hydroxyl; and

y is an integer selected from 1, 2, 3, 4, 5, 6, 7, 8, 9 and 10.

In yet another embodiment, the disclosure provides for compound having the structure of Formula III(a):

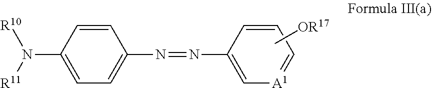

or a pharmaceutically acceptable salt, solvate, polymorph, or prodrug thereof, wherein,

A¹ is selected from N, or CR¹⁶;

R¹⁰ and R¹¹ are individually selected from H or a (C₁-C₆) alkyl;

R¹⁷ is an ether or polyether having the structure of

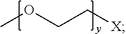

X is a radiohalogen; and

y is an integer selected from 1, 2, 3, 4, 5, 6, 7, 8, 9 and 10.

In a further, the disclosure provides for a compound having the structure of:

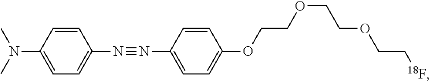

or a pharmaceutically acceptable salt, solvate, polymorph, or prodrug thereof.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The compounds of the disclosure may also be provided as a prodrug, which is a functional derivative of the multi-targeting agent and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The compounds disclosed herein may further comprise additional targeting ligands, like ligands which target the compounds to the brain. Examples of such ligands, include peptides derived from rabies viral glycoprotein (RVG) (e.g., see Cui et al., Immunity & Aging 16:10 (2019), the disclosure of which is incorporated herein in its entirety).

It has been found in the studies presented herein that the compounds of the disclosure have utility as imaging agents for neurodegenerative brain tissue. In particular, it was found that the compounds of the disclosure selectively bound to post mortem brain tissue from subjects who had Alzheimer's disease and Parkinson's disease vs. control subjects. Further, by incorporating radiohalogens (e.g., ¹⁸F, ¹²⁵I, ²¹¹At, ¹²³I, ¹²⁴I, and ^{76/77/78}Br), the compounds of the disclosure are especially useful for imaging with high specific radioactivity and selectivity to neurodegenerative brain tissue. As such, these radiolabeled compounds of the disclosure are ideally suited for detecting and monitoring the progression of a neurodegenerative disorder. Examples of neurodegenerative disorders include, but are not limited to, Alzheimer's disease, Parkinson's disease, prion disease, motor neuron diseases, Huntington's disease, spinocerebellar ataxia, spinal muscular atrophy, amyotrophic lateral sclerosis, Friedreich's ataxia, and Lewy body disease. In a particular embodiment, the radiolabeled compounds of the disclosure are used to image neurodegeneration in subjects with Alzheimer's disease or Parkinson's disease. The tissue selectivity is capable of further enhancement by coupling this highly selective radiolabeled compound with targeting agents, such as microparticles or brain targeting ligands. Methods to image the location and/or binding activity of radiolabeled compounds of the disclosure can used standard imaging techniques, such as, positron emission tomography (PET) imaging, single photon emission computed tomography (SPECT), magnetic resonance imaging, or autoradiography.

PET is a non-invasive imaging technique that uses radioactive isotopes to map chemical or metabolic activity in living organisms. PET is commonly used to diagnose and monitor cancers, brain disorders and disease. It has also been an important research tool for investigating chemical and functional processes in the areas of biochemistry, biology, physiology, anatomy, molecular biology, and pharmacology. While traditional radiography and three-dimensional imaging techniques, such as x-ray computed tomography (CT) and magnetic resonance imaging (MRI), provide structural information, PET scanning provides physiological information of metabolic activity leading to biochemical changes that generally occur long before the associated structural changes can be detected by the more traditional imaging techniques.

Positrons are positively charged electrons emitted by the nucleus of an unstable radioisotope. The radioisotope is unstable because it is positively charged and has too many protons. Release of the positron stabilizes the radioisotope by converting a proton into a neutron. For radioisotopes used in PET, the element formed from positron decay is stable. All radioisotopes used in PET decay by positron emission. The positron travels a small distance, which depends on its energy, before combining with an electron during a so-called “annihilation”. The annihilation of the positron and electron converts the combined mass into two gamma rays that are emitted at 180° to each other along a so-called “line of coincidence”. These gamma rays are readily detectable outside the human body by the detectors of the tomograph. The coincidence lines provide a detection scheme for forming the tomographic image.

Several radioisotopes are commonly used for PET including ₁₁C, ¹⁸F, ¹⁵O, and ¹³N. The radioactive isotope that becomes a source of gamma rays for PET is first chemically incorporated into a compound disclosed herein. The compound, therefore, can act as a “tracer” for neurodegeneration, which is then administered to the patient, typically by injection or inhalation. The more neurodegeneration, the more compound that is bound. Accordingly, a compound of the disclosure can not only detect neurodegeneration but also can be used to quantitate the amount of neurodegeneration based upon the PET scans. Moreover, the rate of neurodegeneration, or any improvement thereof, can be determined based upon taking PET scans of the patient over multiple time points using a compound of the disclosure.

Like other clinical imaging scanners, the typical PET scanner consists of detectors surrounding the subject to be imaged. The detectors are coupled to a scintillator, which converts gamma rays to light photons. The light photons are then converted into electrical impulses. Each electrical impulse generated at a detector corresponds to an “event”, or the arrival at the detector of a gamma-ray photon that originated at an annihilation within the subject.

Examples of scintillator materials for gamma-ray detection include, but are not limited to, sodium iodide crystal, bismuth germinate (BGO), and barium fluoride (BaF₂). An example of detectors includes photomultiplier tubes.

In a typical PET scanner, each detector communicates with the CPU via independent data links, each of which is dedicated to a particular channel. The detector area commonly limits the spatial resolution obtainable in the reconstructed tomographic image. Therefore, to obtain good spatial resolution, it is not unusual for a PET scanner to be comprised of thousands of detectors with an equally large number of corresponding channels and data links.

It was further found in the studies presented herein, that the compounds of the disclosure were highly selective inhibitors of monoamine oxidases (MAOs). In particular it was found that FAZIN3 selectively bound to MAO-A by use of known MAO inhibitors clorgyline (MAO-A inhibitor) and deprenyl (MAO-B inhibitor). MAO inhibitors have found use in treating depression and movement disorders. Accordingly, it is expected that the non-radiolabeled compounds of the disclosure would find a similar therapeutic use. As such, the disclosure further provides for the use of the non-radiolabeled compounds of the disclosure for the treatment of subjects with depression or movement disorders. Examples of depression or movement disorders, include, but are not limited to, Parkinson's disease, major depressive disorder, depression, or Attention deficit hyperactivity disorder. Effective doses can be determined based upon in vivo studies in animal models.

The disclosure further provides for a pharmaceutical composition comprising a compound of the disclosure. Such pharmaceutical compositions may comprise physiologically acceptable surface-active agents, carriers, diluents, excipients, smoothing agents, suspension agents, film forming substances, and coating assistants, or combinations thereof. Acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

Techniques for formulation and administration of the compositions described herein may be found in “Remington's Pharmaceutical Sciences,” Mack Publishing Co., Easton, Pa., 18th edition, 1990.

Suitable routes of administration of the pharmaceutical composition may, for example, include oral, rectal, transmucosal, topical, or intestinal administration; parenteral delivery, including intramuscular, subcutaneous, intravenous, intramedullary injections, as well as intrathecal, direct intraventricular, intraperitoneal, intranasal, or intraocular injections. The pharmaceutical composition can also be administered in sustained or controlled release dosage forms, including depot injections, osmotic pumps, pills, transdermal (including electrotransport) patches, and the like, for prolonged and/or timed, pulsed administration at a predetermined rate.

The pharmaceutical compositions of the disclosure may be manufactured in a manner that is itself known, e.g., by means of conventional mixing, dissolving, granulating, dragee-making, levigating, emulsifying, encapsulating, entrapping or tableting processes.

Pharmaceutical compositions for use as described herein thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized.

For transmucosal administration, penetrants appropriate to the barrier to be permeated may be used in the formulation.

Pharmaceutical formulations for parenteral administration, e.g., by bolus injection or continuous infusion, include aqueous solutions of the compounds in water-soluble form. Additionally, suspensions of the compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the compounds can be formulated readily by combining the compounds with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a patient to be treated. Pharmaceutical preparations for oral use can be obtained by combining the compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active multi-targeting agent doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insulator may be formulated containing a powder mix of the compound and a suitable powder base such as lactose or starch.

Further disclosed herein are various pharmaceutical compositions well known in the pharmaceutical art for uses that include intraocular, intranasal, and intra-auricular delivery. Suitable penetrants for these uses are generally known in the art. Pharmaceutical compositions for intraocular delivery include aqueous ophthalmic solutions of the compounds in water-soluble form, such as eyedrops, or in gellan gum (Shedden et al., Clin. Ther., 23(3):440-50 (2001)) or hydrogels (Mayer et al., Opthalmologica, 210(2):101-3 (1996)); ophthalmic ointments; ophthalmic suspensions, such as microparticulates, drug-containing small polymeric particles that are suspended in a liquid carrier medium (Joshi, A., J. Ocil. Pharmacol., 10(1):29-45 (1994)), lipid-soluble formulations (Alm et al., Prog. Clin. Biol. Res., 312:447-58 (1989)), and microspheres (Mordenti, Toxicol. Sci., 52(1):101-6 (1999)); and ocular inserts. All of the above-mentioned references, are incorporated herein by reference in their entireties. Such suitable pharmaceutical formulations are most often and preferably formulated to be sterile, isotonic and buffered for stability and comfort. Pharmaceutical compositions for intranasal delivery may also include drops and sprays often prepared to simulate in many respects nasal secretions to ensure maintenance of normal ciliary action. As disclosed in Remington's Pharmaceutical Sciences, 18th Ed., Mack Publishing Co., Easton, Pa. (1990), which is incorporated herein by reference in its entirety, and well-known to those skilled in the art, suitable formulations are most often and preferably isotonic, slightly buffered to maintain a pH of 5.5 to 6.5, and most often and preferably include antimicrobial preservatives and appropriate drug stabilizers. Pharmaceutical formulations for intra-auricular delivery include suspensions and ointments for topical application in the ear. Common solvents for such aural formulations include glycerin and water.

The pharmaceutical composition may also be formulated in rectal compositions such as suppositories or retention enemas, e.g., containing conventional suppository bases such as cocoa butter or other glycerides.

In addition to the pharmaceutical formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For hydrophobic compounds, a suitable pharmaceutical carrier may be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethyl sulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Compounds intended to be administered intracellularly may be administered using techniques well known to those of ordinary skill in the art. For example, the compounds may be encapsulated into liposomes. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. The liposome may be coated with a tissue-specific antibody. The liposomes will be targeted to and taken up selectively by the desired organ. Alternatively, small hydrophobic organic molecules may be directly administered intracellularly.

For use in applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more compounds of the disclosure, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The disclosure further provides that the methods and compositions described herein can be further defined by the following aspects (aspects 1 to 55):

1. A compound having the structure of Formula (I) or Formula (II):

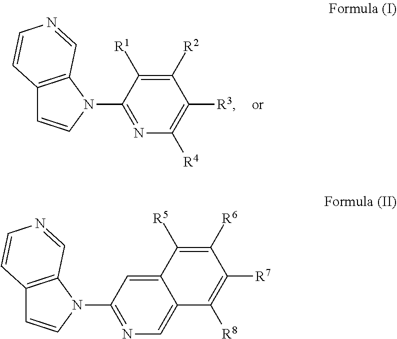

or a pharmaceutically acceptable salt, solvate, polymorph, or prodrug thereof, wherein,

R¹-R⁴ are each individually selected from H, —OR⁹, a halogen, a radiohalogen, a hydroxyl, an amino, an alkoxy, an azide, a ketone, a carboxyl, a carboxylate, an aldehyde, a boronic acid, a boronic ester, a haloformyl, an imide, a nitrile, an isonitrile, a nitro, a thiol, a sulfinyl, a sulfo, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆) hetero-alkenyl, an optionally substituted (C₁-C₆)hetero-alkynyl, an optionally substituted (C₄-C₁₂)cycloalkyl, an aryl, and a heterocycle group, wherein at least one of R¹-R⁴ is —OR⁹;

R⁵-R⁸ are each individually selected from H, —OR⁹, a halogen, a radiohalogen, a hydroxyl, an alkoxy, an azide, a ketone, a carboxyl, a carboxylate, an aldehyde, a boronic acid, a boronic ester, a haloformyl, an imide, a nitrile, an isonitrile, a nitro, a thiol, a sulfinyl, a sulfo, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆) hetero-alkenyl, an optionally substituted (C₁-C₆)hetero-alkynyl, an optionally substituted (C₄-C₁₂)cycloalkyl, an aryl, and a heterocycle group, wherein at least one of R⁵-R⁸ is —OR⁹ or a radiohalogen;

R⁹ is an ether or polyether having the structure of

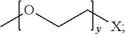

X is a halogen, a radiohalogen, or a hydroxyl; and

y is an integer selected from 1, 2, 3, 4, 5, 6, 7, 8, 9 and 10.

2. The compound of aspect 1, wherein R¹-R⁴ is selected from —OR⁹ and H, and one of R¹-R⁴ is —OR⁹.

3. The compound of aspect 1 or aspect 2, wherein R¹, R³ and R⁴ are H, and R² is —OR⁹.

4. The compound of aspect 1, wherein R¹-R⁴ is selected from —OR⁹, a radiohalogen, and H, and wherein one of R¹-R⁴ is —OR⁹ and one of R¹-R⁴ is ¹²⁵I.

5. The compound of aspect 1, wherein R¹ and R⁴ are H, R² is —OR⁹, and R³ is a radiohalogen.

6. The compound of aspect 5, wherein R³ is ¹²⁵I.

7. The compound of aspect 1, wherein R⁵-R⁸ is selected from ²¹¹At and H, and one of R⁵-R⁸ is a radiohalogen.

8. The compound of aspect 7, wherein R⁵, R⁷, and R⁸ are H, and R⁶ is a radiohalogen.

9. The compound of aspect 8, wherein R⁶ is ¹²⁵I or ²¹¹At.

10. The compound of aspect 1, wherein R⁵-R⁸ is selected from —OR⁹ and H, and one of R⁵-R⁸ is —OR⁹.

11. The compound of aspect 1, wherein X is a radiohalogen selected from ¹⁸F, ¹²⁵I, ²¹¹At, ¹²³I, ¹²⁴I, ^{76/77/78}Br.

12. The compound of aspect 11, wherein X is ¹⁸ F.

13. The compound of aspect 1, wherein the compound comprises the structure of:

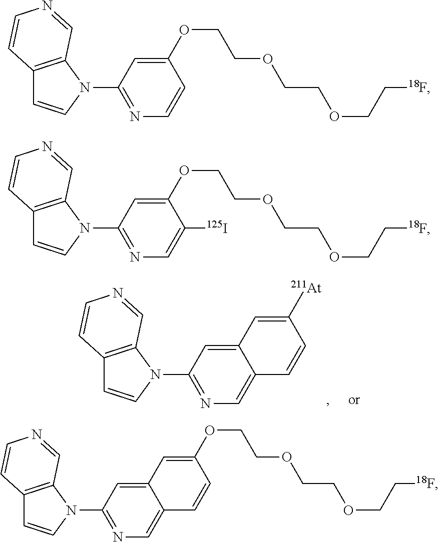

or a pharmaceutically acceptable salt, solvate, polymorph, or prodrug thereof.

14. A method of imaging neurodegeneration in a postmortem brain specimen, comprising:

contacting a postmortem brain specimen from a subject who has a neurodegenerative disorder or is suspected of having a neurodegenerative disorder with a compound of any one of aspects 1 to 13,

detecting and/or quantitating binding of the compound to brain tissue comprising neurodegeneration using an imaging technique that detects radioactivity emitted by the compound.

15. The method of aspect 14, wherein the imaging technique is selected from positron emission tomography (PET) imaging, single photon emission computed tomography (SPECT), magnetic resonance imaging, or autoradiography.

16. The method of aspect 15, wherein the imaging technique is positron emission tomography (PET) imaging.

17. The method of any one of aspects 14 to 16, wherein the subject has or is suspected of having neurodegenerative disorder selected from the group consisting of Alzheimer's disease, Parkinson's disease, prion disease, motor neuron diseases, Huntington's disease, spinocerebellar ataxia, spinal muscular atrophy, amyotrophic lateral sclerosis, Friedreich's ataxia, and Lewy body disease.

18. The method of any one of aspects 14 to 17, wherein the neurodegeneration is caused by an accumulation of neurofibrillary tangles (NFT).

19. A pharmaceutical composition comprising a compound of any one of aspects 1 to 13 and a pharmaceutically acceptable carrier, diluent, and/or excipient, wherein the compound comprises a radiohalogen.

20. The pharmaceutical composition of aspect 19, wherein the pharmaceutical composition is formulated for oral or parenteral delivery.

21. A method for imaging neurodegeneration in a subject having a neurodegenerative disorder or suspected of having a neurodegenerative disorder, comprising:

administering the pharmaceutical composition of any one of aspects 14 to 20 to the subject,

detecting and/or quantitating binding of the compound to brain tissue comprising neurodegeneration using an imaging technique that detects radioactivity emitted by the compound.

22. The method of aspect 21, wherein the subject has neurodegenerative disorder selected from the group consisting of Alzheimer's disease, Parkinson's disease, prion disease, motor neuron diseases, Huntington's disease, spinocerebellar ataxia, spinal muscular atrophy, amyotrophic lateral sclerosis, Friedreich's ataxia, and Lewy body disease.

23. The method of aspect 22, wherein the subject has Alzheimer's disease or Parkinson's disease.

24. The method of any one of aspects 21 to 23, wherein the imaging technique is selected from positron emission tomography (PET) imaging, single photon emission computed tomography (SPECT), magnetic resonance imaging, or autoradiography.

25. The method of aspect 24, wherein the imaging technique is positron emission tomography (PET) imaging.

26. The method of any one of aspects 21 to 25, wherein the neurodegeneration is caused by an accumulation of neurofibrillary tangles (NFT).

27. A compound having the structure of Formula (III):

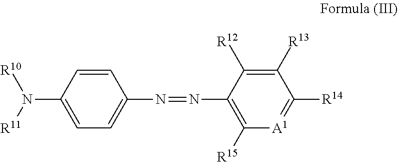

or a pharmaceutically acceptable salt, solvate, polymorph, or prodrug thereof, wherein,

A¹ is selected from N, or CR¹⁶;

R¹⁰ and R¹¹ are individually selected from H or a (C₁-C₆) alkyl;

R¹²-R¹⁶ are each individually selected from H, —OR¹⁷, a halogen, a radiohalogen, a hydroxyl, an amino, an alkoxy, an azide, a ketone, a carboxyl, a carboxylate, an aldehyde, a boronic acid, a boronic ester, a haloformyl, an imide, a nitrile, an isonitrile, a nitro, a thiol, a sulfinyl, a sulfo, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆) hetero-alkenyl, an optionally substituted (C₁-C₆)hetero-alkynyl, an optionally substituted (C₄-C₁₂)cycloalkyl, an aryl, and a heterocycle group, wherein at least one of R¹-R⁴ is —OR¹⁷;

R¹⁷ is an ether or polyether having the structure of

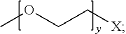

X is a halogen, a radiohalogen or a hydroxyl; and

y is an integer selected from 1, 2, 3, 4, 5, 6, 7, 8, 9 and 10.

28. The compound of aspect 27, wherein R¹⁴ is —OR¹⁷, and R¹²-R¹³ are H.

29. The compound of aspect 27 or aspect 28, wherein A¹ is CR¹⁶, and R¹⁶ is an H.

30. The compound of any one of aspects 27 to 29, wherein X is a radiohalogen selected from ¹⁸F, ¹²⁵I, ²¹¹At, ¹²³I, ¹²⁴I, and ^{76/7/78}Br.

31. The compound of any one of aspects 27 to 30, wherein X is ¹⁸F_{.}

32. The compound of any one of aspects 27 to 31, wherein the compound has the structure of:

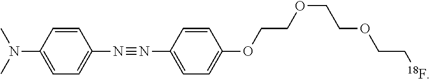

33. The compound of aspect 27, wherein R¹⁴ is a radiohalogen, and R¹²-R¹³ are H.

34. The compound of aspect 33, wherein A¹ is CR¹⁶, and R¹⁶ is an H.

35. The compound of aspect 33 or aspect 34, wherein R¹⁴ is a radiohalogen selected from ¹⁸F, ¹²⁵I, ²¹¹At, ¹²³I, ¹²⁴I, and ^{76/77/78}Br.

36. The compound of aspect 35, wherein R¹⁴ is ¹²⁵I or ¹²⁴I.

37. A method of imaging neurodegeneration in a postmortem brain specimen, comprising:

contacting a postmortem brain specimen from a subject who has a neurodegenerative disorder or is suspected of having a neurodegenerative disorder with a compound of any one of aspects 27 to 36,

detecting and/or quantitating binding of the compound to brain tissue comprising neurodegeneration using an imaging technique that detects radioactivity emitted by the compound.

38. The method of aspect 37, wherein the imaging technique is selected from positron emission tomography (PET) imaging, single photon emission computed tomography (SPECT), magnetic resonance imaging, or autoradiography.

39. The method of aspect 38, wherein the imaging technique is positron emission tomography (PET) imaging.

40. The method of any one of aspects 37 to 39, wherein the subject has or is suspected of having neurodegenerative disorder selected from the group consisting of Alzheimer's disease, Parkinson's disease, prion disease, motor neuron diseases, Huntington's disease, spinocerebellar ataxia, spinal muscular atrophy, amyotrophic lateral sclerosis, Friedreich's ataxia, and Lewy body disease.

41. The method of any one of aspects 37 to 40, wherein the neurodegeneration is caused by an accumulation of neurofibrillary tangles (NFT).

42. A pharmaceutical composition comprising a compound of any one of aspects 27 to 36, and a pharmaceutically acceptable carrier, excipient, and/or diluent.

43. The pharmaceutical composition of aspect 42, wherein the pharmaceutical composition is formulated for oral or parenteral delivery.

44. A method for imaging neurodegeneration in a subject having a neurodegenerative disorder or suspected of having a neurodegenerative disorder, comprising:

administering the pharmaceutical composition of aspect 42 or aspect 43 to the subject,

detecting and/or quantitating binding of the compound to brain tissue comprising neurodegeneration using an imaging technique that detects radioactivity emitted by the compound.

45. The method of aspect 44, wherein the subject has neurodegenerative disorder selected from the group consisting of Alzheimer's disease, Parkinson's disease, prion disease, motor neuron diseases, Huntington's disease, spinocerebellar ataxia, spinal muscular atrophy, amyotrophic lateral sclerosis, Friedreich's ataxia, and Lewy body disease.

46. The method of aspect 45, wherein the subject has Alzheimer's disease.

47. The method of any one of aspects 44 to 46, wherein the imaging technique is selected from positron emission tomography (PET) imaging, single photon emission computed tomography (SPECT), magnetic resonance imaging, or autoradiography.

48. The method of aspect 47, wherein the imaging technique is positron emission tomography (PET) imaging.

49. The method of any one of aspects 44 to 48, wherein the neurodegeneration is caused by an accumulation of amyloid β (Aβ).

50. A method for inhibiting the activity of a monoamine oxidase comprising:

contacting the monoamine oxidase with a compound of aspect 1, or a pharmaceutical composition thereof, wherein the compound has the structure of Formula I:

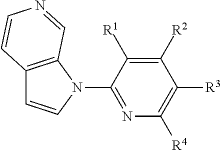

or a pharmaceutically acceptable salt, solvate, polymorph, or prodrug thereof, wherein,

R¹-R⁴ are each individually selected from H, —OR⁹, a halogen, a hydroxyl, an amino, an alkoxy, an azide, a ketone, a carboxyl, a carboxylate, an aldehyde, a boronic acid, a boronic ester, a haloformyl, an imide, a nitrile, an isonitrile, a nitro, a thiol, a sulfinyl, a sulfo, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆) hetero-alkenyl, an optionally substituted (C₁-C₆)hetero-alkynyl, an optionally substituted (C₄-C₁₂)cycloalkyl, an aryl, and a heterocyclyl, wherein at least one of R¹-R⁴ is —OR⁹;

R⁹ is an ether or polyether having the structure of

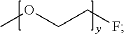

and

y is an integer selected from 1, 2, 3, 4, 5, 6, 7, 8, 9 and 10.

51. The method of aspect 50, wherein the monoamine oxidase is contacted with the compound in vitro.

52. The method of aspect 50, wherein the monoamine oxidase is contacted with the compound in vivo.

53. The method of any one of aspects 50 to 52, wherein the monoamine oxidase is monoamine oxidase-A.

54. A method to treat a subject suffering from depression or a movement disorder, comprising:

administering to the subject an effective amount of a compound having the structure of Formula I:

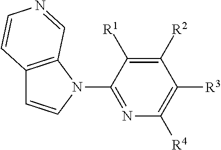

or a pharmaceutically acceptable salt, solvate, polymorph, or prodrug thereof, wherein,

R¹-R⁴ are each individually selected from H, —OR⁹, a halogen, a hydroxyl, an amino, an alkoxy, an azide, a ketone, a carboxyl, a carboxylate, an aldehyde, a boronic acid, a boronic ester, a haloformyl, an imide, a nitrile, an isonitrile, a nitro, a thiol, a sulfinyl, a sulfo, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆) hetero-alkenyl, an optionally substituted (C₁-C₆)hetero-alkynyl, an optionally substituted (C₄-C₁₂)cycloalkyl, an aryl, and a heterocycle, wherein at least one of R¹-R⁴ is —OR⁹;

R⁹ is an alkoxy having the structure of

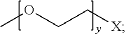

X is F; and

y is an integer selected from 1, 2, 3, 4, 5, 6, 7, 8, 9 and 10.

55. The method of aspect 54, wherein the subject has Parkinson's disease, major depressive disorder, depression, or Attention deficit hyperactivity disorder.

The following examples are intended to illustrate but not limit the disclosure. While they are typical of those that might be used, other procedures known to those skilled in the art may alternatively be used.

### EXAMPLES

All compressed gases were supplied by Airgas, Inc. Aqueous hydrogen [¹⁸F]Fluoride was purchased from PETNET Solutions. [³H]PIB was purchased from American Radiolabeled Chemicals, Inc., St. Louis, Mo. and [¹²⁵I]IPPI was prepared as reported previously (Mukherjee et al., 2020). Iodine-125 radioactivity was counted in a Capintec CRC-15R dose calibrator while low level counting was carried out in a Capintec Caprac-R well-counter. FAZIN compounds and [¹⁸F]-FAZIN compounds were prepared as described herein. Specialty chemicals were obtained from 1Click Chemistry, New Jersey, MK-6240 and 6-azaindole were purchased from AbaChemScene, New Jersey. Clorgyline and (R)-deprenyl were purchased from Research Biochemicals (Sigma Aldrich, St Louis, Mo.). All other chemicals were obtained commercially from Sigma Aldrich, St. Louis, Mo. All solvents used were provided by Fisher Scientific. For QC chemical purity, a Waters or Gilson HPLC system with UV detector set at 280 nm was used with 4.6×250 mm C18 Econosil reverse-phase analytical column was used. A Semi-preparative HPLC column 100×250 mm 10μ Econosil C18 reverse-phase was used.

Analytical thin-layer chromatography (TLC) was used to monitor reactions (Baker-flex, Phillipsburg, N.J., USA). RadioTLC were scanned on an AR-2000 imaging scanner (Eckart & Ziegler, Berlin, Germany). Electrospray mass spectra were obtained from a Model 7250 mass spectrometer (Micromass LCT). Proton NMR spectra were recorded on a Bruker OM EGA 500-MHz spectrometer. Tritium was assayed by using a Packard Tri-Carb Liquid scintillation counter with 65% efficiency. Human postmortem brain tissue samples were obtained from Banner Sun Health Research Institute, Sun City, Ariz. brain tissue repository for in vitro experiments. Age and gender matched AD brain and cognitively normal (CN) brain tissue samples selected for end-stage pathology (Braak & Braak stage of VI; Braak and Braak 1991). Human postmortem brain slices were obtained from chunks of frozen tissue on a Leica 1850 cryotome cooled to −20° C. Tritium and iodine-125, and fluorine-18 autoradiographic studies were carried out by exposing tissue samples on storage phosphor screens (Perkin Elmer Multisensitive, Medium MS and tritium sensitive phosphor screens). The apposed phosphor screens were read and analyzed by OptiQuant acquisition and analysis program of the Cyclone Storage Phosphor System (Packard Instruments Co., Boston, Mass.). All postmortem human brain studies were approved by the Institutional Biosafety Committee of University of California, Irvine.

General Synthesis of the FAZIN series of compounds. chloropyridinol (2-chloropyridin-3-ol, 2-chloropyridin-4-ol, 6-chloropyridin-2-ol, 6-chloropyridin-3-ol) is pegylated in the presence of a strong nonnucleophilic base (e.g., NaO^{t}Bu) in a polar aprotic solvent (e.g., THF) to provide for a pegylated chloropyridine intermediate, which is then coupled with 1H-pyrrolo[2,3-c]pyridine in presence of a strong nonnucleophilic base (e.g., NaO^{t}Bu) in a polar aprotic solvent (e.g., THF) and a palladium catalyst (e.g., t-BuXPhos palladium (II)biphenyl-2-amine mesylate) to form a pegylated pyrrrolo-pyridine intermediate that is then converted to the fluorinated end product by reacting the pegylated pyrrrolo-pyridine intermediate with diethylaminosulfur trifluoride (DAST) in a polar aprotic solvent (e.g., dichloromethane).

Synthesis of FAZIN3. 2-Chloro-4-O-HydroxyPEG3-Pyridine: As shown in FIG. 6, 2-Chloro-4-hydroxy pyridine (136 mg) was treated with sodium tert-butoxide (140 mg) in tetrahydrofuran (THF) at 60° C. for 30 minutes. To this solution 2-[2-(2-bromoethoxy)ethoxy]ethanol (220 mg) was added and the reaction was heated at 65-70° C. for 24 h. The mixture was then cooled, THF evaporated and 10 mL water added and organics were extracted using dichloromethane (CH₂Cl₂). The CH₂Cl₂ layer was dried with anhydrous magnesium sulfate and purified using preparative TLC (CH₂Cl₂:CH₃OH 9:1) to provide 2-chloro-4-O-hydroxyPEG₃-pyridine, as an amber oil 60% yield. Mass Spectra: [M+H]⁺ 262, 100%; [M+Na]⁺ 284, 25%.

4-O-HydroxyPEG3-2-(1H-pyrrolo[2,3-c]pyridine-1-yl)pyridine: 6-Azaindole, (100 mg, 0.85 mmol) was treated with potassium tert-butoxide (100 mg, 0.93 mmol) in dimethylformamide (DMF, 2 mL) for 15 mins at 100° C. Catalytic amounts of palladium catalyst (t-BuXPhos palldium (II)biphenyl-2-amine mesylate) was used. After which, 2-chloro-4-O-hydroxyPEG3-pyridine (261 mg, 1 mmol) was added to the reaction mixture and this mixture was then heated at 100° C. for 24 h. The mixture was then cooled, 10 mL water added and organics were extracted using dichloromethane (CH₂Cl₂). The CH₂Cl₂ layer was dried with anhydrous magnesium sulfate and purified using preparative TLC (CH₂Cl₂:CH₃OH 9:1) to provide a light brown oil, 4-O-HydroxyPEG3-2-(1H-pyrrolo[2,3-c]pyridine-1-yl)pyridine in 30% yield. Mass Spectra: [M+H]⁺ 344, 100%.

4-O-FluoroPEG3-2-(1H-pyrrolo[2,3-c]pyridine-1-yl)pyridine, FAZIN3: 4-O-HydroxyPEG3-2-(1H-pyrrolo[2,3-c]pyridine-1-yl)pyridine, (10 mg) was taken up in dichloromethane (2 mL) and treated with diethylaminosulfur trifluoride (5 μL) at 25° C. and allowed to stir for 24 h. The mixture was then washed with saturated sodium bicarbonate, water added and organics were extracted using dichloromethane (CH₂Cl₂). The CH₂Cl₂ layer was dried with anhydrous magnesium sulfate and purified using preparative TLC (CH₂Cl₂:CH₃OH 9:1) to provide a light brown oil, 4-O-fluoroPEG3-2-(1H-pyrrolo[2,3-c]pyridine-1-yl)pyridine (FAZIN3) in 50% yield. Mass Spectra: [M+H]⁺ 346, 100%.

4-O -TosyloxyPEG3-2-(1H-pyrrolo[2,3-c]pyridine-1-yl)pyridine: 4-O-HydroxyPEG3-2-(1H-pyrrolo[2,3-c]pyridine-1-yl)pyridine, (35 mg) was taken up in dichloromethane (5 mL) and one equivalent of pyridine was added. To this stirring solution at ambient temperature, toluenesulfonyl chloride (20 mg) was added at 25° C. and allowed to stir for 24 h. The mixture was then washed with saturated sodium bicarbonate, water added and organics were extracted using dichloromethane (CH₂Cl₂). The CH₂Cl₂ layer was dried with anhydrous magnesium sulfate and purified using preparative TLC (CH₂Cl₂:CH₃OH 9:1) to provide a light brown oil, 4-O-tosyloxyPEG₃-2-(1H-pyrrolo[2,3-c]pyridine-1-yl)pyridine (AZIN3-Otosylate) in 50% yield. Mass Spectra: [M+H]⁺ 499, 100%.

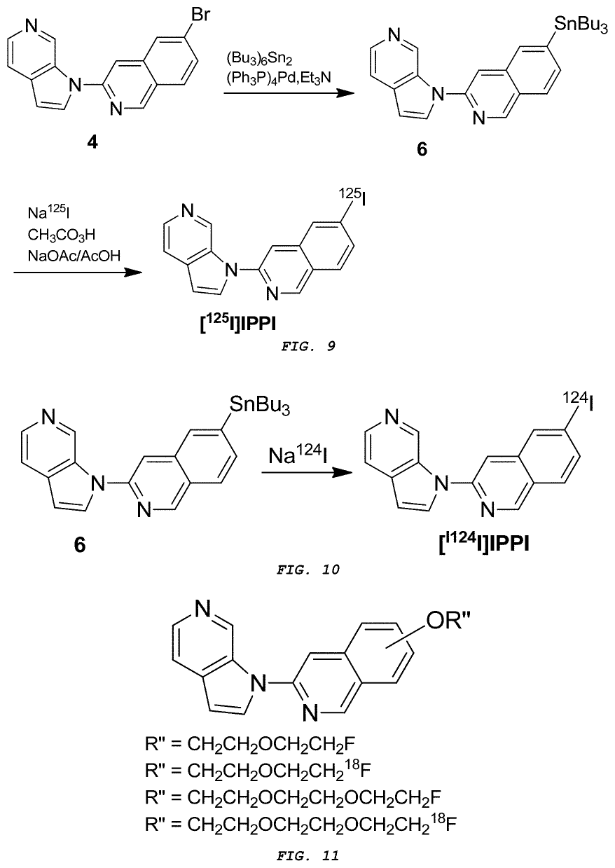

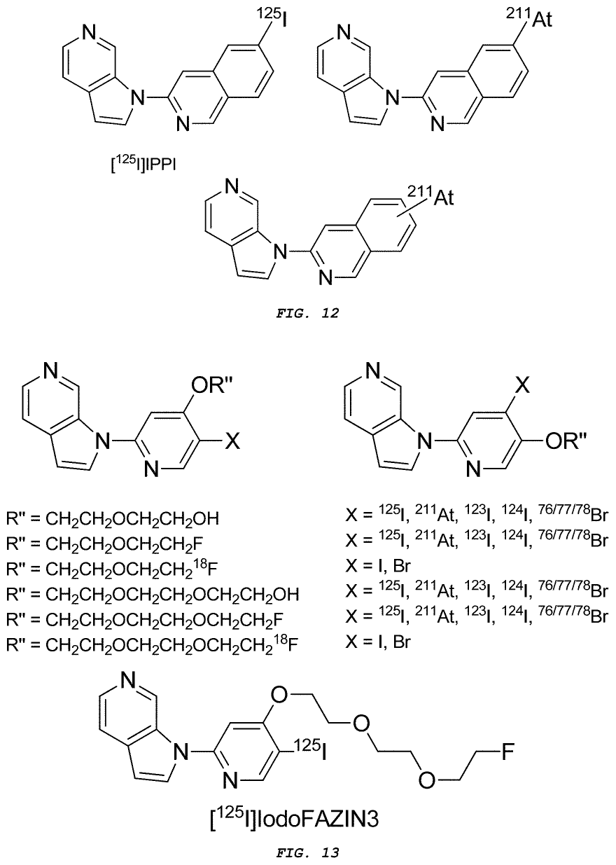

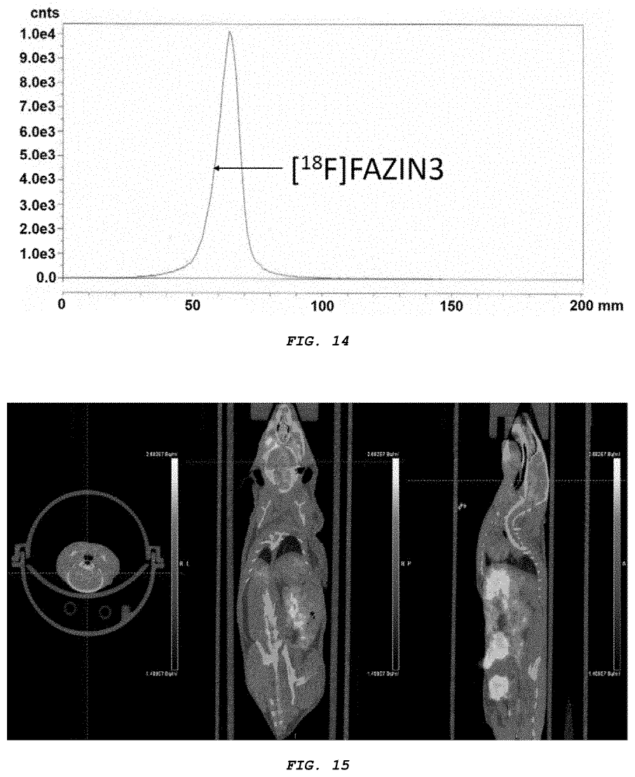

Radiosynthesis of [¹⁸F]FAZIN3. 4-O-[¹⁸F]FluoroPEG3-2-(1H-pyrrolo[2,3-c]pyridine-1-yl)pyridine: As shown in FIG. 9, The radiosynthesis of [¹⁸F]FAZIN3 was carried out using an automated chemistry processing computer unit (CPCU). Fluorine-18 in H₂¹⁸O from a PETNET cyclotron was passed through a QMA-light sep-pak (Waters Corp. Milford, Mass.), preconditioned with 3 mL of K₂CO₃, 140 mg/mL, followed by 3 mL of anhydrous acetonitrile. The fluorine-18 trapped in the QMA-light sep-pak was then eluted with 1 mL Kryptofix222/K₂CO₃ (360 mg/75 mg in 1 mL of water and 24 mL of acetonitrile) and transferred to the CPCU reaction vessel. The “SYNTH1” program in the CPCU was used for the synthesis. This involved initial drying of the ¹⁸F-fluoride, Kryptofix, and K₂CO₃mixture at 120° C. for 10 mins. Subsequently, acetonitrile (2mL) from CPCU reagent vial #2 was added and evaporated at 120° C. for 7 mins to ensure dryness of the ¹⁸F-fluoride mixture. Following this, the precursor, AZIN3-Otosylate (1-2 mg in 0.5 mL of anhydrous acetonitrile continued in CPCU reagent vial #3) was added and the reaction went for 15 to 30 min at 96° C. Subsequent to the reaction, CH₃OH (7 mL contained in CPCU reagent vial #4) was added to the mixture and the CH₃OH contents were passed through a neutral alumina sep-pak (prewashed with methanol) in order to remove any unreacted ¹⁸F-fluoride. The collected CH₃OH solution coming out of the CPCU now contained [¹⁸F]FAZIN3. The CH₃OH was removed in vacuo and the residue was taken up for HPLC purification. Product was purified in a reverse-phase HPLC C₁₈ Econosil column 250×10 mm (Alltech Assoc. Inc., Deerfield, Ill.) with 60% acetonitrile: 40% water containing 0.1% triethylamine with a flow rate of 2.5 mL/min. The retention time of [¹⁸F]FAZIN3 was found to be 10.5 minutes. The [¹⁸F]FAZIN3 fraction was collected into a flask and the solvent was removed in vacuo using a rotary evaporator to dryness. The radiosynthesis was accomplished in 1.5 h with an overall radiochemical yield of 20-40% decay corrected. Specific activity was measured to be greater than 2000 Ci/mmol.

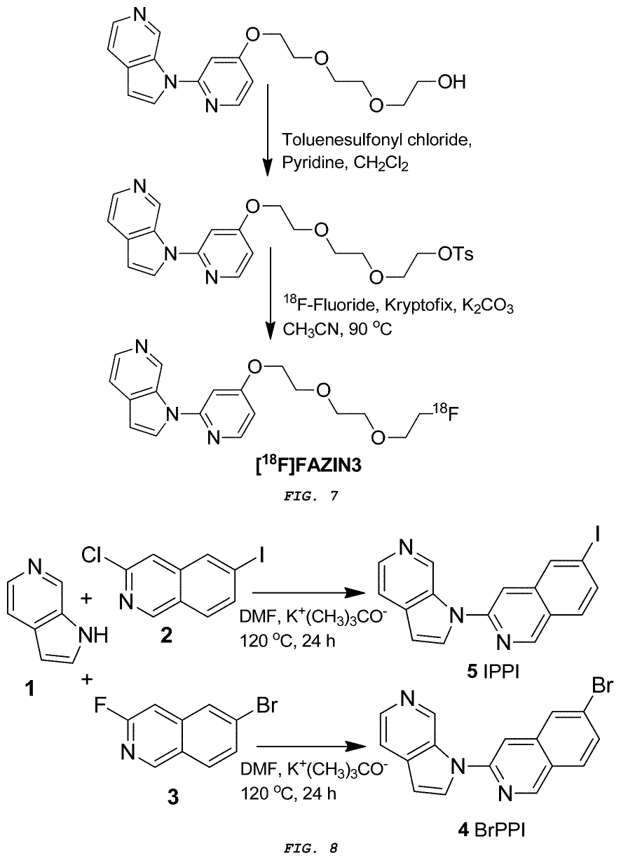

Synthesis of 6-Iodo-3-(1H-pyrrolo[2,3-c]pyridine-1-yl)isoquinoline (IPPI). As shown in FIG. 8, 6-Azaindole 1(88 mg, 0.75 mmol) was treated with potassium tert-butoxide (115 mg, 1.03 mmol) in dimethylformamide (DMF, 3 mL) for 15 mins at 120° C. Subsequently, 2-chloro-6-iodo-isoquinoline, 2 (289 mg, 1 mmol) was added to the reaction mixture and this mixture was then heated at 125° C. for 24 hrs. The mixture was then cooled, 10 mL water added and organics were extracted using dichloromethane (CH₂Cl₂). The CH₂Cl₂ layer was dried with anhydrous magnesium sulfate and purified using preparative TLC (CH₂Cl₂:CH₃OH 9:1) to provide IPPI, as an off-white solid 5 (61 mg, 0.18 mmol) in 24% yield. Spectral properties were similar to reported values (Walji A, et al., 2015). NMR (500 MHz, CDCl₃): δ 10.07 (s, 1H), 8.43 (d, J=5.3 Hz, 1H), 8.22 (s, 1H), 8.19 (s, 1H), 8.01 (dd, J=4.5, 0.86 Hz, 1H), 7.96 (d, J=3.45 Hz, 1H), 7.86 (d, J=8.8 Hz, 1H), 7.66 (s, J=8.8 Hz, 1H), 7.61 (dd, J=4.5, 0.86 Hz, 1H), 6.80 (d, J=3.4 Hz, 1H). Mass Spectra (ESI): 372 ([M+H]⁺, 100%), 743 ([2M+H]⁺, 100%).

Synthesis of 6-Bromo-3-(1H-pyrrolo[2,3-c]pyridine-1-yl)isoquinoline (BrPPI). As shown in FIG. 8, 6-Azaindole 1(100 mg, 0.85 mmol) was treated with potassium tert-butoxide (100 mg, 0.93 mmol) in dimethylformamide (DMF, 2 mL) for 15 mins at 120° C. Subsequently, 2-fluoro-6-bromo-isoquinoline 3 (100 mg, 0.44 mmol) was added to the reaction mixture and this mixture was then heated at 125° C. for 24 hrs. The mixture was then cooled, 10 mL water added and organics were extracted using dichloromethane (CH₂Cl₂). The CH₂Cl₂ layer was dried with anhydrous magnesium sulfate and purified using preparative TLC (CH₂Cl₂:CH₃OH 9:1) to provide a light brown solid, BrPPI 4 (50 mg, 0.15 mmol) in 34% yield. NMR (500 MHz, CDCl₃) : δ 9.62 (s, 1H), 9.27 (s, 1H), 8.39 (d, J=5.39 Hz, 1H), 8.08 (s, 1H), 8.01 (dd, J=4.5, 0.86 Hz, 1H), 7.91 (d, J=8.8 Hz, 1H), 7.72 (d, J=8.8 Hz, 1H), 7.69 (d, J=8.8 Hz, 1H), 7.61 (d, J=5.37 Hz, 1H), 6.78 (d, J=3.4 Hz, 1H). Mass Spectra (ESI): 324 ([M+H]⁺, 100%), 326 ([M+H]⁺, 97%).

Synthesis of 6-Tributyltin-3-(1H-pyrrolo[2,3-c]pyridine-1-yl)isoquinoline. As shown in FIG. 9, to a solution of BrPPI 4 (11 mg; 34 μmol) in anhydrous triethylamine (1 mL) under nitrogen, bistributyltin (60 mg; 103 μmol) and Tetrakis(triphenylphosphine)palladium(0) (5 mg; 4.3 μmol) were added. This reaction mixture was held at reflux overnight at 88° C. The dark yellow crude reaction mixture was purified over prep silica gel TLC plate using hexane:ethyl acetate 1:1 as a solvent. The title product with Rf=0.5 was isolated (BrPPI Rf=0.3) to yield pure tributyltin product 6 (4.5 mg; 8.4 μmol) as an oil in 25% yield. NMR (500 MHz, CDCl₃): δ 9.65 (s, 1H), 9.25 (s, 1H), 8.38 (d, J=5.49 Hz, 1H), 8.07 (s, 1H), 8.05 (s, 1H), 7.97 (d, J=8.0 Hz, 1H), 7.80 (s, 1H), 7.72 (d, J=7.4 Hz, 1H), 7.66 (d, J=5.4 Hz, 1H), 6.81 (d, J=1.8 Hz, 1H) , 1.60 (m, 3H) , 1.38 (m, 2H) , 1.19 (t, J=8 Hz, 2H), 0.92 (t, J=7.3 Hz, 3H). Mass Spectra (ESI): 534 ([M+H]⁺, 40%), 535 ([M+H]⁺, 80%), 537 ([M+H]⁺, 100%).

Radiosynthesis of 6-[¹²⁵I]iodo-3-(1H-pyrrolo[2,3-c]pyridine-1-yl)isoquinoline, [¹²⁵I]IPPI: Radioiodination hood (CBS Scientific, Inc) placed inside a fume hood designated to handle radioactive materials was used to carry out all processes for radioionation, separation and purification. Iodine-125 radiolabeling was carried out by using modification of our previously reported radiolabeling procedures with iodine-123 and iodine-124 (Pandey et al., 2012; 2014). No-carrier-added Na¹²⁵I (18.5 MBq, approx. 100 μL 0.01N NaOH, 644 MBq/pg; American Radiolabeled Chemicals, St. Louis) was taken in a V-vial. To this vial 100 μL of 0.1 M sodium acetate/acetic acid buffer, pH 4 was added. This was mixed and then peracetic acid (50 μL, Sigma-Aldrich) was added and allowed to stand for 15 minutes at room temperature. Following this, tributyltin precursor 6 (50 pg) dissolved in 100 μL ethanol was added and the reaction mixture was heated at 70° C. for 0.5 h. The vial was then cooled to room temperature and quenched by adding NaHSO₃ (100 μL of 1 mg/mL aq. stock) and saturated NaHCO₃ solution (200 μL) was added. The reaction mixture was then extracted with CH₂Cl₂ (2×400 μL), dried (MgSO₄), and evaporated (N₂ gas stream) to provide crude [¹²⁵I]IPPI. The crude mixture was purified on preparative TLC (CH₂Cl₂:CH₃OH 9:1) and separated from unreacted starting material. [¹²⁵I]IPPI was separated and extracted using ethanol. RadioTLC of the ethanolic solution (see FIG. 8) showed a purity >95% and Rf=0.7 consistent with reference IPPI. This was ethanolic [¹²⁵I]IPPI at a concentration of 1 MBq/mL was used for in vitro experiments.

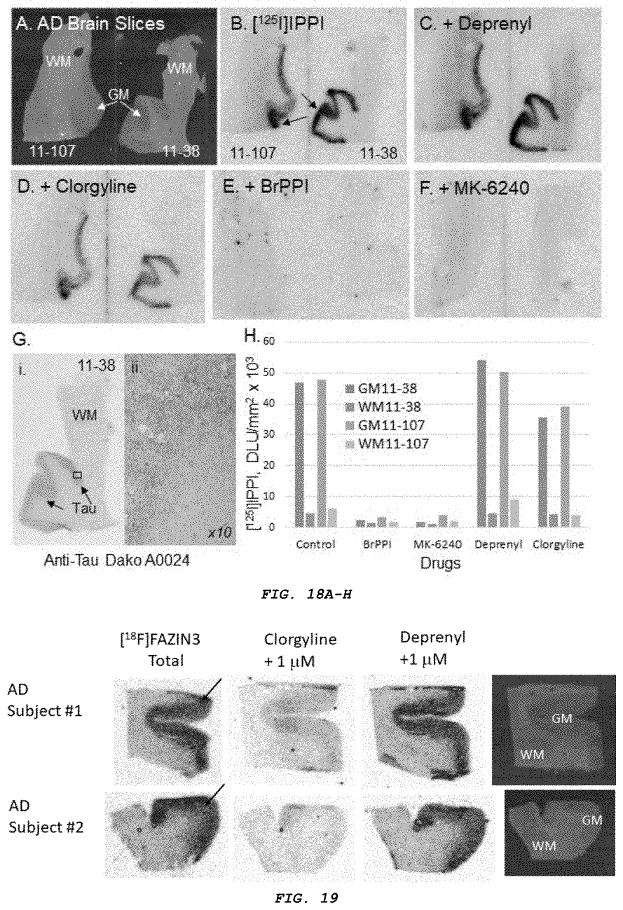

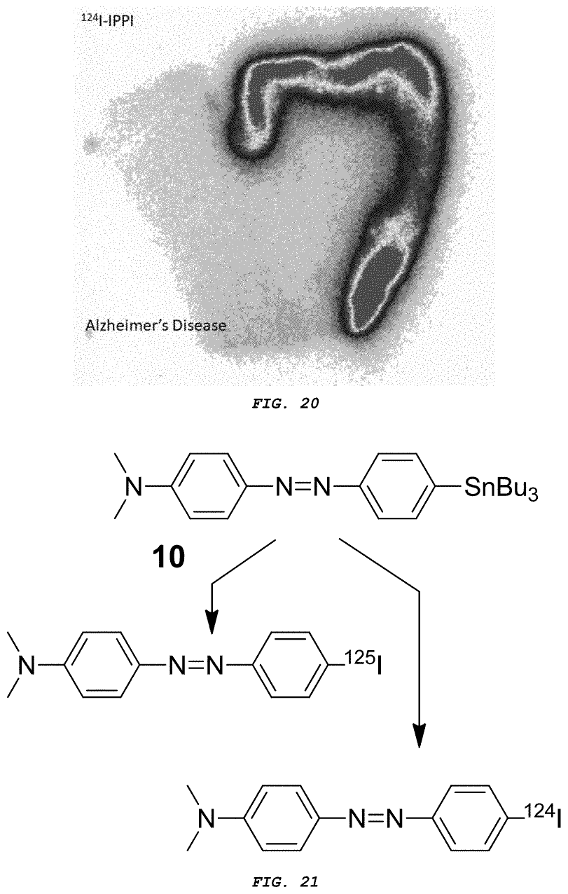

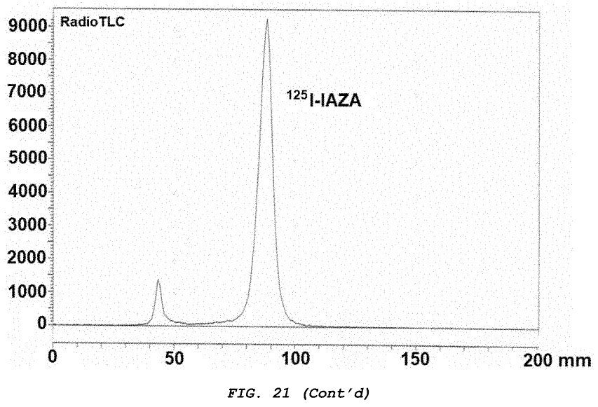

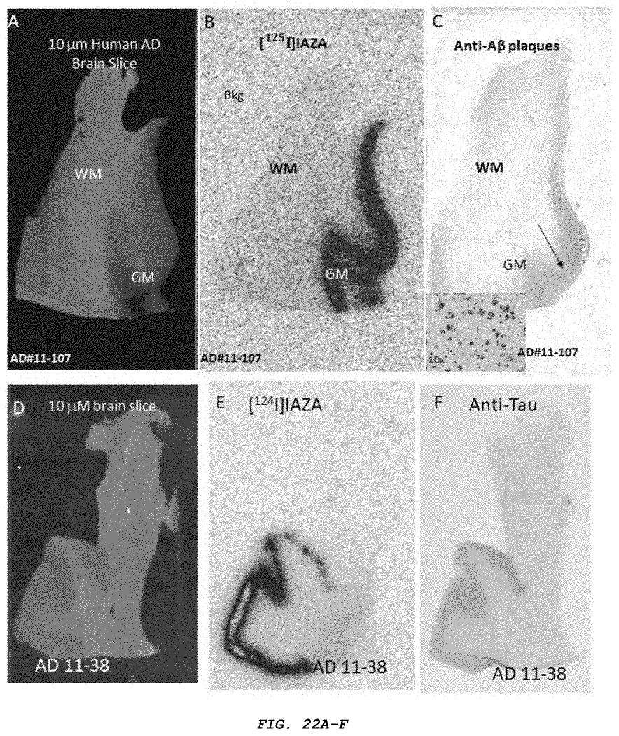

Astatine-211 Derivatives. Excellent binding of [¹²⁵I]IPPI to Tau protein in Alzheimer's disease brains was found (e.g., see FIG. 18). As iodine can be replaced with other halogens, the synthesis scheme used to make IPPI presented herein can be modified to replace iodine with another halogen. The halogen Astatine-211 is an Auger electron emitter was used for the generation of additional compounds. [²¹¹At]AtPPI finds use in imaging in vitro as well as in vivo. It is postulated that targeted delivery of [²¹¹At]AtPPI to neurofibrillary tangles will cause the breakdown of neurofibrillary tangles due to the emitted Auger electrons and clearance of the broken down smaller pieces of the neurofibrillary tangle from the brain.

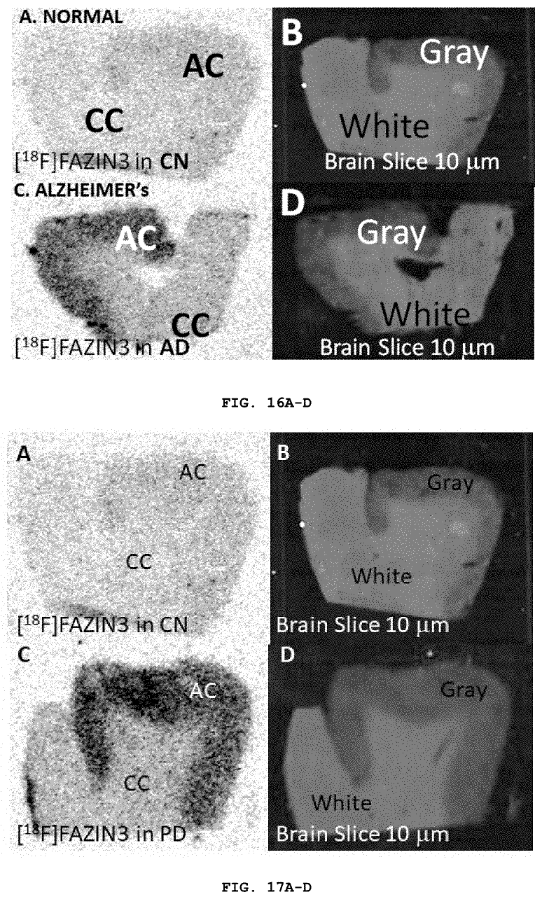

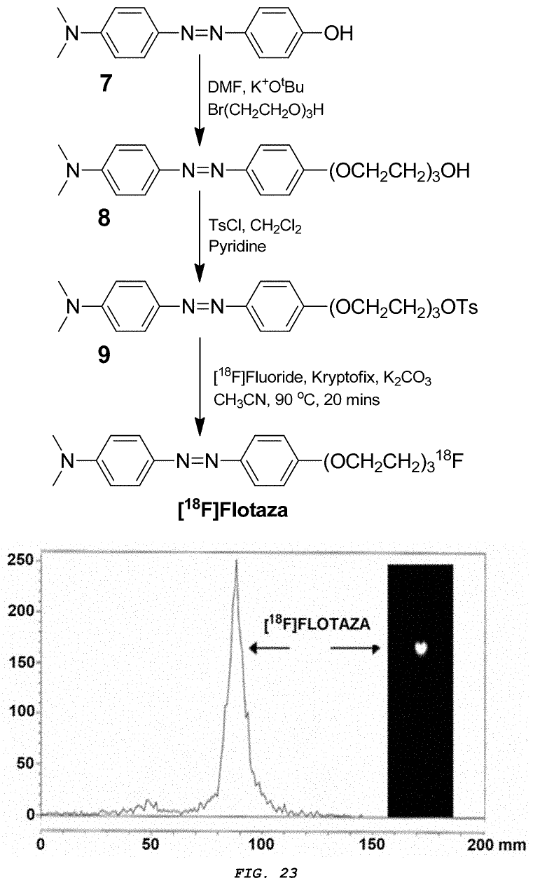

Synthesis of Flotaza. As shown in FIG. 23, starting with 4-hydroxy-4′-dimethylaminoazobenzene 7. Pegylated alcohol 8 was successfully prepared by reacting 7 with bromo-PEG3-alcohol in moderate yields. Reaction of the alcohol 8 with diethylamino sulfur trifluoride (DAST) led to a complex mixture, which may have been due to the high reactivity of DAST. Therefore, the alcohol was first converted to the corresponding tosylate 9, followed by reacting the tosylate with [¹⁸F]fluoride, Kryptofix and potassium carbonate (K₂CO₃) in acetonitrile (CH₃CN) to provide [¹⁸F]Flotaza.

[¹⁸F]FAZIN selectively binds strongly to postmortem neurodegenerative brain tissue. Human anterior cingulate sections containing corpus callosum were sectioned from cognitively normal (CN), Alzheimer's disease (AD) and Parkinson's disease (PD) subjects. These sections were used to evaluate the binding of [18F]FAZIN3 to the grey matter (anterior cingulate, AC) and white matter (corpus callosum) in the three groups of postmortem human brain subjects. The slides containing the sections (10 μm thick) were preincubated in PBS buffer for 15 minutes in three separate chambers at 25° C. The preincubation PBS buffer was discarded and to each chamber was then added [¹⁸F]FAZIN3 and 60 mL of PBS buffer for a final concentration of 1 μCi/mL of [¹⁸F]FAZIN3. The chambers were incubated at 25° C. for 1 hour. The slides were then washed with cold PBS buffer twice, 3 minutes each time and cold water for 2 minutes, respectively. The slides with the brain sections were air dried, exposed overnight on a phosphor film, and then placed on the Phosphor Autoradiographic Imaging System/Cyclone Storage Phosphor System (Packard Instruments Co). Regions of interest (ROIs) were drawn on the slices and the extent of binding of [¹⁸F]FAZIN3 was measured in DLU/mm² using the OptiQuant acquisition and analysis program (Packard Instruments Co). It was found, [¹⁸F]FAZIN3 bound strongly in the brains of AD subjects compared to normal subjects (CN) (e.g., see FIG. 16). Additionally, it was found that [¹⁸F]FAZIN3 bound strongly in the brains of Parkinson's Disease subjects compared to normal (CN) (e.g., see FIG. 17). Therefore, [¹⁸F]FAZIN3 selectively binds strongly to neurodegenerative brain tissue.

[¹⁸F]FAZIN is a selective reversible inhibiter of MAO-A. Human anterior cingulate sections containing corpus callosum were sectioned from cognitively normal (CN), Alzheimer's disease (AD) and Parkinson's disease (PD) subjects. These sections were used to evaluate the effect of drugs on the binding of [¹⁸F]FAZIN3. MK-6240 (10 μM) was used since it is known to bind to Tau. Monoamine oxidase (MAO) B inhibitor, (R)-deprenyl was used (1 μM) and MAO A inhibitor, clorgyline was used (1 μM). The slides containing the sections (10 μm thick) were preincubated in PBS buffer for 15 minutes in eight different slide chambers (one total binding and seven with the different drugs). The preincubation PBS buffer was discarded and appropriate amount of each drug (dissolved in ethanol) was added to the chambers with the slides. To each chamber was then added [18F]FAZIN3 and 60 mL of PBS buffer for a final concentration of 1 μCi/mL of [¹⁸F]FAZIN3. The chambers were incubated at 25° C. for 1.25 h. The slides were then washed with cold PBS buffer twice, and cold water, respectively. The slides with the brain sections were air dried, exposed overnight on a phosphor film, and then placed on the Phosphor Autoradiographic Imaging System/Cyclone Storage Phosphor System (Packard Instruments Co). Regions of interest (ROIs) were drawn on the slices and the extent of binding of [¹⁸F]FAZIN3 was measured in DLU/mm² using the OptiQuant acquisition and analysis program (Packard Instruments Co). It was found that clorgyline inhibits more than 80% of [¹⁸F]FAZIN3, while deprenyl and MK-6240 did not have much effect (e.g., see FIG. 19). [¹⁸F]FAZIN3 is therefore a selective MAO-A binding PET radiotracer suitable for studies related to neurodegeneration.

Assessing the binding specificity of Flotaza. In vitro binding affinity of Flotaza in human AD brain slices using [³H]PIB for Aβ plaques and [¹²⁵I]IPPI for Tau were carried out. The affinity of Flotaza was marginally weaker than TAZA for Aβ plaques (Ki=1.68 nM for Flotaza versus 0.54 nM for TAZA), suggesting that fluoropegylation is well tolerated in the TAZA backbone. Flotaza did not have any significant effect on the binding of [¹²⁵I]IPPI confirming weak Tau binding. Thus, Flotaza is a selective Aβ plaque agent.

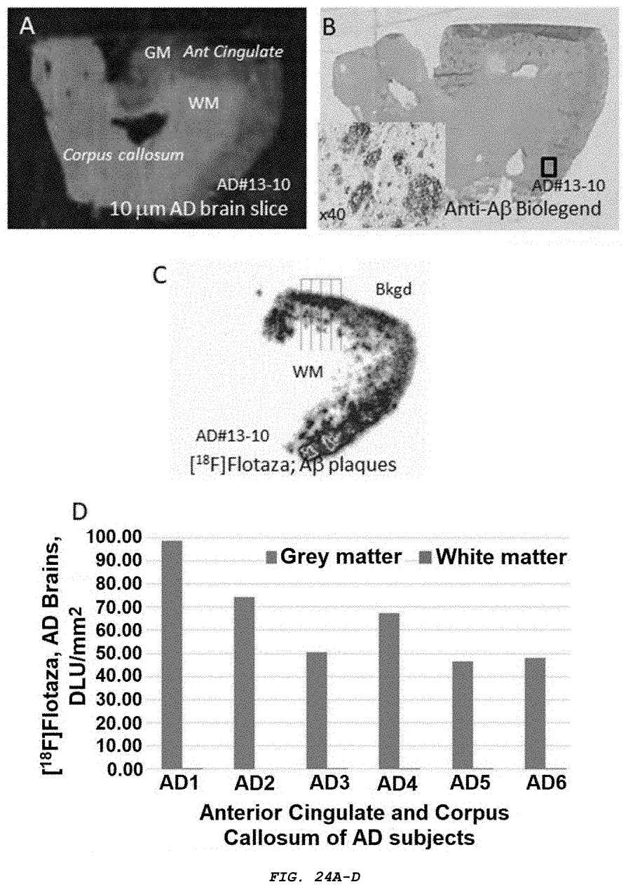

Postmortem human brain autoradiography [¹⁸F]Flotaza in AD subjects. Well characterized brain samples were obtained from Banner Health Research Institute. Brain slices from six AD subjects included anterior cingulate (gray matter, GM) and corpus callosum (white matter, WM) as shown for one subject in FIG. 24A. The AD brain sections of the six AD subjects were further confirmed to contain Aβ plaques in the GM regions by immunostaining with anti-Aβ Biolegend (known to stain Aβ1-16) as shown in FIG. 24B.

Extensive binding of [¹⁸F]Flotaza was seen in the grey matter regions of all the AD subjects. FIG. 24C shows brain slice of one subject with binding of [¹⁸F]Flotaza in the anterior cingulate, while white matter had very little binding. This grey matter binding was significantly reduced when the brain sections were treated with PIB. FIG. 24E shows [¹⁸F]Flotaza binding through the cortical layers, showing greater binding in the outer layers. Similarly, high levels of binding in the gray matter were seen in all the six subjects (FIG. 24D) and was consistent with immunostaining in adjacent sections. White matter binding was very small across all the subjects and ratios between gray matter and white matter was found to be >100 in all the subjects. It must be noted that in these experiments, after [¹⁸F]Flotaza binding, the slices were washed with 50% alcohol in PBS buffer. The white matter binding increased significantly when the slices were washed only with PBS buffer.

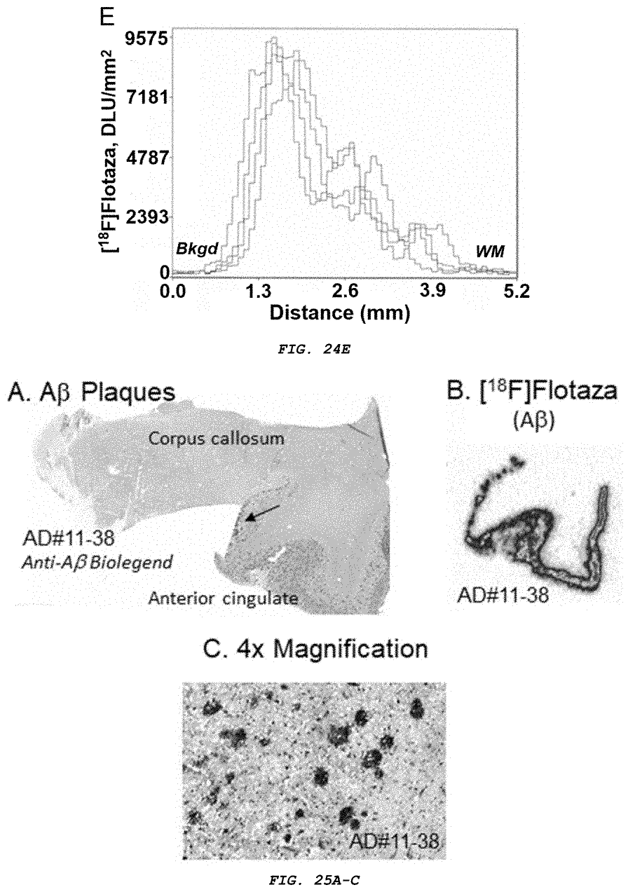

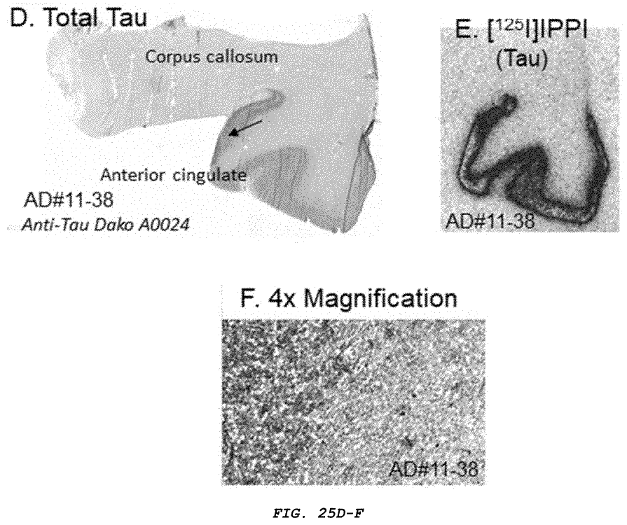

Binding profile of [¹⁸F]Flotaza to Aβ plaques and [¹²⁵I]IPPI to Tau on adjacent brain slices containing anterior cingulate and corpus callosum of the same subject were compared (see FIG. 25). Immunostaining of adjacent slices confirmed the presence of Aβ plaques (see FIG. 25A, C) and Tau (see FIG. 6D, F). Both, [¹⁸F]Flotaza (see FIG. 6B) and [¹²⁵I]IPPI (see FIG. 6E) bound to anterior cingulate extensively in adjacent slices, and was consistent throughout the gray matter regions. This binding is consistent with the immunostaining of the two biomarkers and supports the usefulness of [¹⁸F]Flotaza in the diagnostic use of Aβ plaques in AD.

Notable advantages of Flotaza over other imaging agents. The high degree of binding of [¹⁸F]Flotaza in AD brain slices is similar to the studies with [¹¹C]TAZA. The ratio of gray matter to white matter, however, was significantly higher for [¹⁸F]Flotaza compared to [¹¹C]TAZA. The GM/WM ratios of [¹¹C]TAZA ranged between 20 and 30 in hippocampal AD brain sections. Similarly, [¹¹C]PIB showed lower GM/WM ratios in the hippocampal brain sections, compared to[¹¹C]TAZA. Thus, “AZA” functionality renders unique properties to the molecule yielding higher binding to Aβ plaques. Fluoropegylation is known to reduce lipophilicity of molecules and gives additional advantage to [¹⁸F]Flotaza compared to [¹¹C]TAZA. Molecular modeling analysis of the binding of TAZA and Flotaza revealed very similar binding energies to preferred sites on the Aβ amyloid fibrils.

The olefin analog of the [¹¹C]TAZA, [¹¹C]Dalene exhibited highest amount of white matter binding. Since [¹¹C]Dalene is a close fluoropegylated structural analog of [¹⁸F]florbetaben, the results suggest that [¹⁸F]Flotaza, which is a fluoropegylated analog of [¹¹C]TAZA is likely to yield higher GM/WM ratios compared to [¹⁸F]florbetaben. The most significant structural difference between [¹⁸F]Flotaza and [¹⁸F]florbetaben is the presence of the “AZA” functionality replacing the olefin.

Previous studies with “AZA” group containing PDB derivatives suggested that the “benzothiazole moiety” present in the PDB derivatives may be contributing to their affinity to Tau. Using [¹²⁵I]IPPI labeled brain slices, Flotaza did not have any significant effect on [¹²⁵I]IPPI binding thus suggesting poor affinities of Flotaza for Tau.

It will be understood that various modifications may be made without departing from the spirit and scope of this disclosure.

Accordingly, other embodiments are within the scope of the following claims.

What is claimed is:

1. A compound having the structure of Formula (I) or Formula (II): 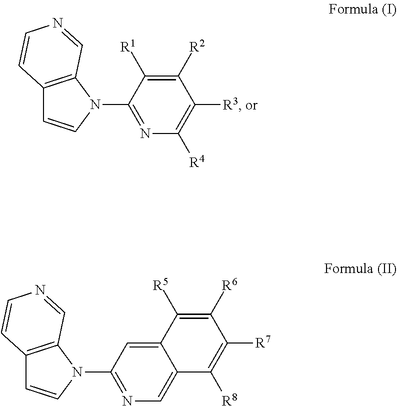
or a pharmaceutically acceptable salt, solvate, polymorph, or prodrug thereof, wherein,
R¹-R⁴ are each individually selected from H, —OR⁹, a halogen, a radiohalogen, a hydroxyl, an amino, an alkoxy, an azide, a ketone, a carboxyl, a carboxylate, an aldehyde, a boronic acid, a boronic ester, a haloformyl, an imide, a nitrile, an isonitrile, a nitro, a thiol, a sulfinyl, a sulfo, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆) hetero-alkenyl, an optionally substituted (C₁-C₆)hetero-alkynyl, an optionally substituted (C₄-C₁₂)cycloalkyl, an aryl, and a heterocycle group, wherein at least one of R¹-R⁴ is —OR⁹;
R⁵-R⁸ are each individually selected from H, —OR⁹, a halogen, a radiohalogen, a hydroxyl, an alkoxy, an azide, a ketone, a carboxyl, a carboxylate, an aldehyde, a boronic acid, a boronic ester, a haloformyl, an imide, a nitrile, an isonitrile, a nitro, a thiol, a sulfinyl, a sulfo, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆) hetero-alkenyl, an optionally substituted (C₁-C₆)hetero-alkynyl, an optionally substituted (C₄-C₁₂)cycloalkyl, an aryl, and a heterocycle group, wherein at least one of R⁵-R⁸ is —OR⁹ or a radiohalogen;
R⁹ is an ether or polyether having the structure of 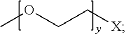
X is a halogen, a radiohalogen, or a hydroxyl; and
y is an integer selected from 1, 2, 3, 4, 5, 6, 7, 8, 9 and 10.

2. The compound of claim 1, wherein R¹-R⁴ is selected from —OR⁹ and H, and one of R¹-R⁴ is —OR⁹.

3. The compound of claim 2, wherein R¹, R³ and R⁴ are H, and R² is —OR⁹.

4. The compound of claim 1, wherein R¹-R⁴ is selected from —OR⁹, a radiohalogen, and H, and wherein one of R¹-R⁴ is —OR⁹ and one of R¹-R⁴ is ¹²⁴I or ¹²⁵I.

5. The compound of claim 1, wherein R¹ and R⁴ are H, R² is —OR⁹, and R³ is a radiohalogen.

6. The compound of claim 5, wherein R³ is ¹²⁴I or ¹²⁵I.

7. The compound of claim 1, wherein R⁵-R⁸ is selected from ²¹¹At and H, and one of R⁵-R⁸ is a radiohalogen.

8. The compound of claim 7, wherein R⁵, R⁷, and R⁸ are H, and R⁶ is a radiohalogen.

9. The compound of claim 8, wherein R⁶ is ¹²⁴I, ¹²⁵I or ²¹¹At.

10. The compound of claim 1, wherein R⁵-R⁸ is selected from —OR⁹ and H, and one of R⁵-R⁸ is —OR⁹.

11. The compound of claim 1, wherein X is a radiohalogen selected from ¹⁸_{F,} ¹²⁵I, ²¹¹At, ¹²³I, ¹²⁴I, and ^{76/77/78}Br.

12. The compound of claim 11, wherein X is ¹⁸F.

13. The compound of claim 1, wherein the compound comprises the structure of: 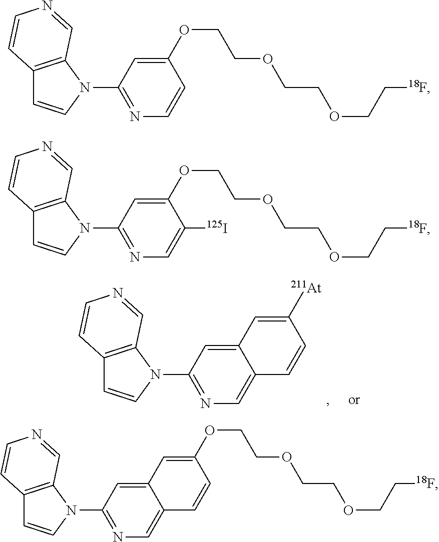
or a pharmaceutically acceptable salt, solvate, polymorph, or prodrug thereof.

14. A method of imaging neurodegeneration in a postmortem brain specimen, comprising:
contacting a postmortem brain specimen from a subject who has a neurodegenerative disorder or is suspected of having a neurodegenerative disorder with a compound of claim 1,
detecting and/or quantitating binding of the compound to brain tissue comprising neurodegeneration using an imaging technique that detects radioactivity emitted by the compound.

15. A pharmaceutical composition comprising a compound of claim 1 and a pharmaceutically acceptable carrier, diluent, and/or excipient, wherein the compound comprises a radiohalogen.

16. The pharmaceutical composition of claim 15, wherein the pharmaceutical composition is formulated for oral or parenteral delivery.

17. A method for imaging neurodegeneration in a subject having a neurodegenerative disorder or suspected of having a neurodegenerative disorder, comprising:
administering the pharmaceutical composition of claim 15 to the subject,
detecting and/or quantitating binding of the compound to brain tissue comprising neurodegeneration using an imaging technique that detects radioactivity emitted by the compound.

18. The method of claim 17, wherein the subject has neurodegenerative disorder selected from the group consisting of Alzheimer's disease, Parkinson's disease, prion disease, motor neuron diseases, Huntington's disease, spinocerebellar ataxia, spinal muscular atrophy, amyotrophic lateral sclerosis, Friedreich's ataxia, and Lewy body disease.

19. The method of claim 17, wherein the imaging technique is selected from positron emission tomography (PET) imaging, single photon emission computed tomography (SPECT), magnetic resonance imaging, or autoradiography.

20. A compound having the structure of Formula (III): 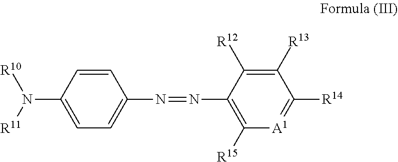
or a pharmaceutically acceptable salt, solvate, polymorph, or prodrug thereof, wherein,
A¹ is selected from N, or CR¹⁶;
R¹⁰ and R¹¹ are individually selected from H or a (C₁-C₆)alkyl;
R¹²-R¹⁶ are each individually selected from H, —OR¹⁷, a halogen, a radiohalogen, a hydroxyl, an amino, an alkoxy, an azide, a ketone, a carboxyl, a carboxylate, an aldehyde, a boronic acid, a boronic ester, a haloformyl, an imide, a nitrile, an isonitrile, a nitro, a thiol, a sulfinyl, a sulfo, an optionally substituted (C₁-C₆)alkyl, an optionally substituted (C₁-C₆)alkenyl, an optionally substituted (C₁-C₆)alkynyl, an optionally substituted (C₁-C₆)hetero-alkyl, an optionally substituted (C₁-C₆) hetero-alkenyl, an optionally substituted (C₁-C₆)hetero-alkynyl, an optionally substituted (C₄-C₁₂)cycloalkyl, an aryl, and a heterocycle group, wherein at least one of R¹-R⁴ is —OR¹⁷;
R¹⁷ is an ether or polyether having the structure of 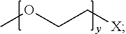
X is a halogen, a radiohalogen or a hydroxyl; and
y is an integer selected from 1, 2, 3, 4, 5, 6, 7, 8, 9 and 10.
